(19) United States
(12) Patent Application Publication
(10) Pub. No.: US 2005/0137555 A1
(43) Pub. Date: Jun. 23, 2005

(54) Individual wrapping container

(75) Inventors: Satoshi Mizutani, Kagawa (JP); Yuki Noda, Kagawa (JP); Wataru Yoshimasa, Kagawa (JP); Megumi Tokumoto, Kagawa (JP)

Correspondence Address: DARBY & DARBY P.C., P. O. BOX 5257, NEW YORK, NY, 10150-5257, US

(73) Assignee: Uni-Charm Corporation, Shikokuchuo-Shi (JP)

(21) Appl. No.: 11/007,811

(22) Filed: Dec. 8, 2004

(30) Foreign Application Priority Data
Dec. 9, 2003 (JP) 2003-411090

(51) Int. Cl. A61F 13/15; A61F 13/20

(52) U.S. Cl. 604/385.02

(57) ABSTRACT

An individual wrapping container according to the present invention prevents deformation and damage of an ineterlabial pad attached between labia accommodated in the individual wrapping container by protecting a peripheral portion of the interlabial pad. The individual wrapping container also prevents contamination of the interlabial pad before use at the time of replacement of the used interlabial pad. The interlabial pad includes the peripheral portion forming a contour of the interlabial pad. The individual wrapping container includes a protective region having higher rigidity than that of the peripheral potion of the interlabial pad accommodated in the individual wrapping container so as to protect the peripheral portion.

### CROSS-REFERENCE TO RELATED APPLICATIONS

This application is based upon and claims the benefit of priority from Japanese Patent application No. 2003411090 filed on Dec. 9, 2003, the entire contents of which is incorporated herein by reference.

### FIELD OF THE INVENTION

The present invention relates to an individual wrapping container in which an interlabial pad is packaged, and more particularly to an individual wrapping container contrived so as to prevent contamination of the interlabial packaged in the container.

### RELATED ART

Currently, an interlabial pad attached between female labia for absorbing menstrual blood is known. The interlabial pad is sanitary and clean since the pad prevents diffusion of menstrual blood and resultant wide contact between the blood and the body by more tightly contacting with the body than a sanitary napkin. Additionally, the pad attached between labia has features that the interlabial pad is more comfortable during use providing excellent sense of fitness since it is smaller in size than the sanitary napkin, and that the interlabial pad gives less feeling of rejection at the time of attachment than a tampon which needs to be inserted into a vagina.

An example of an interlabial pad of this type is shown by an interlabial pad 710 having an elliptic shape as illustrated in FIG. 42. The interlabial pad 710 is folded double along a line extending parallel to a longitudinal direction to provide two substantially symmetrical and semicircular parts. The interlabial pad 710 is packaged in an individual wrapping container 720 made from a thin vinyl sheet (for example, see Patent Reference Nos. 1 and 2).

The interlabial pad 710 disclosed in Patent Reference No. 1 or 2 is entirely covered with the individual wrapping container 720. Thus, the interlabial pad 710 before use can be carried without being contaminated. Particularly, since the individual wrapping container 720 is made from a vinyl sheet which is not permeable to liquid, contamination of the interlabial pad 710 is avoided even when the individual wrapping container 720 is dropped on a lavatory floor.

An interlabial pad described in Patent Reference No. 2 has a surface side sheet and a back side sheet. A semicircular holding portion is provided on the back side sheet. The interlabial pad is folded double along a center line in a longitudinal direction such that two parts of the back side sheet facing to each other, and is accommodated in an individual wrapping container such that the surface side sheet is disposed to contact the inside surface of the individual wrapping container. The individual wrapping container described in the Patent Reference No. 2 has an inside surface and outside surface and is folded along a longitudinal axis direction such that two parts of the inside surface facing to each other. Three sides of the individual wrapping container are joined and one remaining side is re-closably sealed to provide a bag shape. This structure allows a wearer to hold the holding portion of the interlabial pad and take the pad out of the individual wrapping container. Thus, the surface side sheet of the interlabial pad is not contaminated. Additionally, it is possible to accommodate a used interlabial pad in an individual wrapping container from which an interlabial pad before use is taken out and seal the individual wrapping container with the used pad again.

[Patent Reference No. 1] International Publication No. 02/094151 Pamphlet

[Patent Reference No. 2] U.S. Pat. No. 6,131,736

However, in the individual wrapping container disclosed in Patent Reference No. 1, the initial configuration and the sanitary condition of the interlabial pad at the time of manufacture are not maintained in some cases. For example, when the individual wrapping container 720 covering the interlabial pad is folded for carrying, the interlabial pad contained in therein is folded accordingly, resulting in warp of a peripheral portion of the interlabial pad.

On the other hand, in the individual wrapping container described in Patent Reference No. 2, it is necessary to hold a used interlabial pad by one hand and an interlabial pad before use by the other hand, i.e., with both hands being occupied when it is desired to attach the new pad before placing the used pad in the individual wrapping container. Since it is impossible to produce a sufficient space for inserting the interlabial pad by opening labia using an unoccupied hand and attach the new interlabial pad into labia using the other hand, the new interlabial pad is not securely positioned at an appropriate location. As a result, the absorption performance of the interlabial pad is not sufficiently offered.

### SUMMARY OF THE INVENTION

The present invention has been developed to solve the above-described problems. It is an object of the invention to provide an individual wrapping container capable of preventing contamination of an interlabial pad before use and maintaining an initial condition at the time of manufacture. More specifically, it is an object of the invention to provide an individual wrapping container capable of inhibiting and avoiding load applied on a peripheral portion of the interlabial pad so as to prevent deformation and damage of the interlabial pad accommodated within the individual wrapping container. It is also an object of the invention to provide an individual wrapping container capable of preventing contamination of the interlabial pad before use at the time of replacement of a used interlabial pad. It is further an object of the invention to provide an outer wrapping container and an intermediate wrapping container capable of inhibiting and avoiding load applied on the peripheral portion of the interlabial pad.

More specifically, the followings are provided according to the present invention.

(1) An individual wrapping container in which an interlabial pad attached between labia is packaged, wherein: the interlabial pad includes a peripheral portion forming a contour of the interlabial pad; and the individual wrapping container includes a protective region having higher rigidity than the rigidity of the peripheral potion of the interlabial pad so as to protect the peripheral portion.

The term “rigidity” as described above refers to capability of an object for resisting breakage due to bend, distortion or other cause. The term “peripheral potion” refers to an end portion forming a contour of the interlabial pad. For example, when the interlabial pad is structured such that an absorption member is sandwiched between two sheets to be wrapped therein, the peripheral portion is located in an area where the sheets are connected to each other outside the absorption member where the absorption member is not provided. The peripheral portion is easily deformed when coming into contact with the skin of a wearer.

The individual wrapping container of the invention includes the protective region having higher rigidity than that of the peripheral portion of the interlabial pad. The protective region prevents warp and crush of the peripheral portion of the interlabial pad accommodated in the protective region since the protective region is not easily bent or twisted when shock is applied to the individual wrapping container. Thus, deterioration of wearing comfortableness at the time of attachment of the interlabial pad which is taken out of the individual wrapping container is avoided. Additionally, since the protective region is provided, the peripheral portion of the interlabial pad itself is allowed to be soft. Thus, the interlabial pad offers more enhanced wearing comfortableness. Furthermore, the entire wrapping container may be formed by the protective region having higher rigidity than that of the peripheral portion in the present invention. In this case, the individual wrapping container itself is not easily crushed while being carried, thereby preventing warp and crush of the entire peripheral portion of the interlabial pad accommodated within the individual wrapping container.

When the connected area of the sheets is positioned at the peripheral portion in the interlabial pad as described above, the connection between the sheets at the peripheral portion of the interlabial pad is sometimes separated and one of the sheets is thus warped due to folding of the individual wrapping container or other cause. As a result, the absorption member tends to be exposed, and fibers of the absorption member sometimes remain on the inner wall of the labia directly contacting the absorption member when the wearer attaches the interlabial pad in such a condition. However, it is possible to prevent this problem in advance in the present invention.

(2) The individual wrapping container according to (1), wherein: the peripheral portion of the interlabial pad includes a vestibular floor contact region contacting with a vestibular floor of a wearer during attachment of the interlabial pad; and the protective region is disposed at least in a portion where the individual wrapping container contacts the vestibular floor contact region when the interlabial pad is packaged in the individual wrapping container.

The term “vestibular floor” as described above refers to the region at the base of the right and left labia, that is, the region along a line from the clitoris to the ostium vaginae located in the inner portion of the labia.

In the individual wrapping container of the invention, the protective region is disposed in the vestibular floor contacting region of the interiabial pad contacting the sensitive vestibular floor. This structure particularly prevents deformation and damage of the vestibular floor contacting region of the interlabial pad, thereby avoiding damage to the clitoris and anus caused by attaching the interlabial pad with the area contacting the sensitive clitoris and anus being warped.

(3) The individual wrapping container according to (1) or (2), wherein the protective region is made from a rigid material having predetermined rigidity.

In the individual wrapping container of the invention, the protective region is made from the rigid material having predetermined rigidity. Examples of the rigid material having predetermined rigidity involve polyethylene, polypropylene, polyethylene terephthalate, polyvinyl alcohol, polylactate, polybutyl succinate, nonwoven fabrics and paper, and laminated nonwoven fabrics and paper, each of which is 15 to 60 μm in thickness. More specifically, a film formed by mixing low density polyethylene (LDPE) resin at a rate of 80% or less and high density polyethylene (HDPE) resin at a rate of 20% or more and adjusting the basis weight per unit area in a range from 15 to 35 g/m² may be used. Additionally, a film drawn for enhancing resin orientation may be employed. Furthermore, the individual wrapping container preferably blocks off the color of menstrual blood absorbed by the interlabial pad. For this purpose, pigment may be mixed in a range from 0.2% to 10%, or ink and the like may be printed.

The entire individual wrapping container may be made from the above material, or a sheet made from the above material may be affixed on the protective region.

(4) The individual wrapping container according to (1) through (3), wherein the protective region is embossed.

In the individual wrapping container of the invention, the protective region can be easily provided at an appropriate position by embossing the protective region. Moreover, an additional component is not required for forming the protective region, thereby lowering the material cost. Shown below is a specific example of an embossing pattern in which minute convexes are provided on a film. The height of the minute convexes is in a range from 0.05 to 2 mm, and the distance between the respective minute convexes is in a range from 0.3 to 20 mm. A wavy embossing pattern may be adopted. Particularly when the wavy shape is directed in the lateral direction and continuously formed in the longitudinal direction, a front end 10a and a rear end 10b contacting the vestibular floor can be further protected since buckling strength of the protective region in the longitudinal direction becomes extremely high.

(5) The individual wrapping container according to any one of (1) through (4), wherein: the individual wrapping container is constituted by a sheet body; and the protective region is formed in a folded portion formed by folding a part of the sheet body.

The individual wrapping container of the invention is constituted by a sheet body, and the thickness of the sheet body in the protective region is increased to enhance its strength by folding a part of the sheet body. Since the protective region is formed by folding a single successive sheet of the sheet body, an additional component such as a plurality of sheet bodies to be laminated to each other is not required. Thus, the protective region can be securely provided at an appropriate position. Additionally, the buckling strength of the two-folded sheet is larger than that of two sheets simply overlapped, and external pressure applied is difficult to escape from the folded portion, which further increases the buckling strength.

(6) The individual wrapping container according to any one of (1) through (5), wherein a buckling strength of the protective region is established in a range from 100 mN to 2,000 mN.

In the individual wrapping container of the invention, the buckling strength of the protective region is established in a range from 100 mN to 2,000 mN. When the buckling strength of the protective region is smaller than 100 mN, the peripheral portion as well as the individual wrapping container crushes. When the buckling strength of the protective region is larger than 2,000 mN, it is difficult to wrap the interlabial pad in the individual wrapping container during the wrapping process of the production line due to its extremely high rigidity, which results in output of defective products. In the present invention, however, the interlabial pad can be easily packaged in the individual wrapping container during the wrapping process while protecting the shape of the peripheral portion of the interlabial pad accommodated in the individual wrapping container by establishing the buckling strength in the above-described range. More specifically, a film formed by mixing low density polyethylene at a rate of 50% and high density polyethylene at a rate of 50% and adjusting the basis weight per unit area at 25 g/m² is folded once to produce the protective region having four layers (two layers on one side with the interlabial pad disposed in the middle). In this case, the buckling strength of the protective region is 1,400 mN.

(7) The individual wrapping body comprising an individual wrapping container according to any one of (1) through (6) and a interlabial pad accommodated in the individual wrapping container, wherein: the interlabial pad includes a peripheral portion forming a contour of the interlabial pad; and a buckling strength of the peripheral portion of the interlabial pad is established in a range from 50 mN to 400 mN.

In the individual wrapping body of the invention, the buckling strength of the peripheral portion of the interlabial pad is established in a range from 50 mN to 400 mN. When the buckling-strength of the peripheral portion is smaller than 50 mN, the peripheral portion is easily compressed and bent more than necessary. As a result, the peripheral portion tends to be lumped, thereby deteriorating wearing comfortableness. When larger than 400 mN, the peripheral portion is not deformed more than necessary when the peripheral portion comes into contact with the skin and thus wearing comfortableness cannot be offered to the wearer. In the present invention, however, it is possible to prevent warp of the peripheral portion of the interlabial pad during the wrapping process while maintaining preferable contact with the labia by establishing the buckling strength in the above-described range.

(8) The individual wrapping container in which an interlabial pad attached between labia is packaged, comprising a protective sheet for preventing contamination of the interlabial pad before use, and a discard sheet used for discarding a used interlabial pad, wherein: the protective sheet directly covers the interlabial pad before use to form an inner wrapping body and the discard sheet covers the inner wrapping body when the interlabial pad is packaged in the individual wrapping container; and a temporary closing member for releasably fixing the inner wrapping body and the discard sheet is provided on a surface where the inner wrapping body contacts the discard sheet and/or a surface where the discard sheet contacts the inner wrapping body.

The term “temporary closing member” as described above refers to a component for limiting free motions of the interlabial pad such that the interlabial pad is not shifted from an appropriate position where the interlabial pad is originally disposed within the individual wrapping container.

The individual wrapping container of the invention includes the protective sheet and the discard sheet. The protective sheet directly covers the interlabial pad before use to form the inner wrapping body and the discard sheet covers the inner wrapping body when the interlabial pad is packaged in the individual wrapping container. This structure allows the used interlabial pad to be wrapped in the discard sheet in which the pad before use has been packaged so as to discard the used pad. On the other hand, since the interlabial pad before use is packaged in the protective sheet, the interlabial pad before use can be attached in a clean condition without being contaminated by dirt, dust, germs etc. adhering thereto during discard of the used interlabial pad. Additionally, it is possible to wrap the used interlabial pad in the discard sheet which is non-permeable to liquid and discard it into a sanitary container, and subsequently to discard the empty protective sheet into a toilet closet immediately after taking the interlabial pad before use out of the protective sheet. It is also possible to reduce the possibilities of contamination of the surface of the interlabial pad before use when the wearer stooped to discard the used interlabial pad into the sanitary container and contamination of clothes and the body of the wearer and places inside the lavatory by menstrual blood which flows out of the vagina opening of the wearer during discarding the protective sheet.

The temporary closing member for temporarily and releasably fixing the inner wrapping body and the discard sheet is also provided on a surface where the inner wrapping body contacts the discard sheet and/or a surface where the discard sheet contacts the inner wrapping body. The temporary closing member prevents contamination of the surface of the interlabial pad which may be caused when the interlabial pad slips from the protective sheet inclined due to slight impact on or unbalanced placement of the protective sheet which covers the interlabial pad before use.

Moreover, since the individual wrapping container of the invention has a double-structure in which the discard sheet and the protective sheet are separated from each other, the action for separating the discard sheet and the protective sheet is not required. Thus, the steps for attaching the interlabial pad are facilitated compared with the individual wrapping container formed by a single sheet.

(9) The individual wrapping container according to (8), wherein the temporary closing member is releasable to such a degree that the inner wrapping body is not damaged when the inner wrapping body is separated from the discard sheet, and also has sufficient adhesion for re-adhering after separation.

The term “releasable” as described above refers to the capability of peeling the inner wrapping body from the discard sheet. The term “adhesion” refers to the adhesive property sufficient for adhering to a wall, a paper holder or other place.

In the individual wrapping container of the invention, the temporary closing member is releasable to such a degree that the inner wrapping body is not damaged when the inner wrapping body is separated from the discard sheet. This structure allows the inner wrapping body to be separated from the discard sheet without damaging the inner wrapping body, thereby maintaining a clean condition of the interlabial pad before use accommodated in the inner wrapping body without adhesion of dirt, dust, germs etc. to the pad before use. The temporary closing member of the individual wrapping container also has sufficient adhesion for re-adhering after separation. This structure allows the inner wrapping body before use to be affixed to a wall, a paper holder or other place. Since the temporary closing member of the inner wrapping body before use has adhesion sufficient for adhering to a wall, a paper holder or other place, the interlabial pad does not drop due to inclined attachment to a wall, a paper holder or other place or impact given to the inner wrapping body. Additionally, the inner wrapping body can be easily removed from the position where the inner wrapping body is temporarily fixed without breaking the protective sheet which may be caused when larger peeling force than the protective sheet strength is applied.

The protective sheet has a first surface and a second surface on the side where the protective sheet does not contact the interlabial pad, and the adhesive material is applied to either or both of the first and second surfaces. A region of at least 20 by 20 mm square where the adhesive material is not applied is provided on both surfaces of the protective sheet. This region secures a portion for holding the protective sheet covering the interlabial pad (a portion touched by the thumb and the forefinger) by the wearer, and prevents adhesion of the adhesive material on the protective sheet to the hand and resultant difficulty in handling of the protective sheet when the protective sheet is held and temporarily placed or the interlabial pad is taken out of the protective sheet.

(10) An individual wrapping container in which an interlabial pad attached between labia is packaged, wherein: the individual wrapping container is constituted by a wrapping sheet having a breaking line along which a part of the wrapping sheet can be separated; and the part separated along the breaking line becomes a discard sheet used for discarding a used interlabial pad.

The individual wrapping container of the invention is constituted by a single sheet of the wrapping sheet, and a part of the wrapping sheet can be separated. This structure allows the protective sheet for covering the new interlabial pad and the discard sheet used for discarding the used interlabial pad to be separated from each other. Since the discard sheet used for discarding the used interlabial pad is allowed to be separated, it is possible to hold the interlabial pad before use which is kept packaged and the discard sheet and the interlabial pad by one hand and the other, respectively, so as to cut off along the breaking line. Thus, accidental dropping of the interlabial pad before use is prevented at the time of separation.

(11) The individual wrapping container according to (10), wherein: the individual wrapping container includes at least an accommodating space for accommodating an interlabial pad before use and/or a used interlabial pad; and the accommodating space is formed by folding a part of the wrapping sheet where the breaking line is not located.

In the invention, a part of the wrapping sheet is folded to form a pocket in which the interlabial pad is accommodated. While the interlabial pad before use is being accommodated within the accommodating space, there is no possibility of dropping of the interlabial pad before use during separation along the breaking line. When the used interlabial pad is put into the accommodating space, the used pad can be discarded in a clean manner. Additionally, it is possible to provide two accommodating spaces. In the individual wrapping container of the invention which includes the accommodating space for accommodating the interlabial pad before use and an additional accommodating space on an area other than where the accommodating space for the pad before use is provided on the sheet, for example, the used interlabial pad can be accommodated in the additional accommodating space after separating the wrapping sheet into two parts along the breaking line, so that the used pad can be discarded more clean and smoothly.

(12) The individual wrapping container according to (10), wherein: the individual wrapping container includes at least an accommodating space for accommodating an interlabial pad before use and/or a used interlabial pad; and the accommodating space is a space formed between the wrapping sheet and a mini-sheet attached to the wrapping sheet.

In the individual wrapping container of the invention, the additional mini-sheet is overlapped on the wrapping sheet. This structure allows the accommodating space for the interlabial pad to be easily formed by simply sealing the mini-sheet on the upper side such that a space can be formed.

Additionally, the accommodating space may be divided into two to form a part for the interlabial pad before use and the other part for the used interlabial pad by sealing the accommodating space. A closing tape may be also provided at an end on the lateral side of the wrapping sheet where the interlabial pad is not packaged. In this case, the wrapping sheet is rolled from the side where the used interlabial pad is packaged and closed by the closing tape, thereby sealing the wrapping sheet so as to prevent the used interlabial pad from being dropped.

(13) A wrapping body comprising: individual wrapping bodies each including the individual wrapping containers according to any one of (1) through (6) or (8) through (12) and an interlabial pad packaged in the individual wrapping containers, or individual wrapping bodies according to (7) and an outer wrapping container for accommodating a plurality of the individual wrapping bodies, wherein a subdividing intermediate wrapping container for accommodating the two or more individual wrapping bodies to be sealed therein is provided within the outer wrapping container.

The wrapping body of the invention includes the individual wrapping bodies and the outer wrapping container for accommodating a plurality of the individual wrapping bodies. The subdividing intermediate wrapping container for accommodating the two or more individual wrapping bodies to be sealed therein is also provided within the outer wrapping container. Since the intermediate wrapping container, in other word the subdividing package for accommodating the two or more individual wrapping bodies is included in the outer wrapping container, it is possible to carry only this subdividing package. Additionally, when the wearer drops the subdividing package in the lavatory or other place, contamination of the individual wrapping bodies and the interlabial pads within the individual wrapping bodies is prevented.

(14) The wrapping body according to (13), wherein the intermediate wrapping container is disposed such that a clearance is provided between the intermediate wrapping container and an inner wall of the outer wrapping container.

In the wrapping body of the invention, the intermediate wrapping container is disposed such that a clearance is provided between the intermediate wrapping container and an inner wall of the outer wrapping body. When the outer wrapping container is accidentally dropped during conveyance or by the wearer, shock thus caused is absorbed by the clearance, thereby preventing the shock from being transmitted to the intermediate wrapping container. It is also possible to prevent transmission of the shock to the interlabial pad which is sealed in the individual wrapping body accommodated within the intermediate wrapping container.

(15) The wrapping body according to (13) or (14), wherein the intermediate wrapping container is made from an airtight material so that the individual wrapping bodies can be airtight-sealed therein.

In the wrapping body of the invention, the intermediate wrapping container is made from an airtight material so that the individual wrapping bodies can be airtight-sealed in the intermediate wrapping container which is filled with air. When the wearer accidentally drops the intermediate wrapping container in which a plurality of the individual wrapping bodies are sealed, this structure allows shock to be absorbed by air, thereby preventing transmission of the shock to the interlabial pads accommodated in the individual wrapping bodies. Thus, the interlabial pad can be attached without deterioration of the wearing comfortableness when the wearer accidentally drops the intermediate wrapping container in which a plurality of the individual wrapping bodies are sealed.

As described above, the individual wrapping container covering the interlabial pad has higher rigidity than that of the peripheral portion of the interlabial pad according to the present invention. Particularly, a region having high rigidity is provided on a portion covering the peripheral portion so as to prevent frequent warp of the peripheral portion of the interlabial pad accommodated in the individual wrapping container while it is being carried. Accordingly, provided herein is the individual wrapping container capable of preventing warp of the peripheral portion of the interlabial pad contained in the individual wrapping container and separation of the connection between the sheets as covering means. Also provided is the individual wrapping container capable of preventing contamination of the interlabial pad before use at the time of replacement. Further provided are the outer wrapping container and the intermediate wrapping container capable of inhibiting and avoiding load to be applied on the peripheral portion of the interlabial pad.

### BRIEF DESCRIPTION OF THE DRAWINGS

FIG. 1 is a front view of an individual wrapping body in a first embodiment according to the invention.

FIG. 2 illustrates a condition where an individual wrapping container of the individual wrapping body in the first embodiment according to the invention is opened.

FIG. 3 is a perspective view of an interlabial pad in the first embodiment according to the invention.

FIG. 4 is a front view of the interlabial pad in the first embodiment according to the invention.

FIG. 5 is a perspective view of the interlabial pad in the first embodiment according to the invention.

FIG. 6 is a perspective view of the interlabial pad in the first embodiment according to the invention.

FIG. 7 is a front view of an individual wrapping body in a second embodiment according to the invention.

FIG. 8 is a cross-sectional view of the individual wrapping body in the second embodiment according to the invention.

FIG. 9 is a front view of an individual wrapping body in a third embodiment according to the invention.

FIG. 10 is a cross-sectional view of the individual wrapping body in the third embodiment according to the invention.

FIG. 11 is a front view of an individual wrapping body in a fourth embodiment according to the invention.

FIG. 12 is a cross-sectional view of the individual wrapping body in the fourth embodiment according to the invention.

FIG. 13 illustrates a method for measuring buckling strength according to the invention.

FIG. 14 illustrates the method for measuring buckling strength according to the invention.

FIG. 15 illustrates the result obtained by the measuring buckling strength according to the invention.

FIG. 16 is a front view of an individual wrapping body in a fifth embodiment according to the invention.

FIG. 17 is a cross-sectional view of the individual wrapping body in the fifth embodiment according to the invention.

FIG. 18A illustrates the individual wrapping body in the fifth embodiment according to the invention.

FIG. 18B illustrates the individual wrapping body in the fifth embodiment according to the invention.

FIG. 18C illustrates the individual wrapping body in the fifth embodiment according to the invention.

FIG. 18D illustrates the individual wrapping body in the fifth embodiment according to the invention.

FIG. 19A is a perspective view of an inner wrapping body in the fifth embodiment according to the invention.

FIG. 19B is a perspective view of the inner wrapping body in the fifth embodiment according to the invention.

FIG. 20A illustrates steps for wrapping an interlabial pad in the fifth embodiment according to the invention.

FIG. 20B illustrates steps for wrapping the interlabial pad in the fifth embodiment according to the invention.

FIG. 20C illustrates steps for wrapping the interlabial pad in the fifth embodiment according to the invention.

FIG. 21A illustrates an individual wrapping body in a sixth embodiment according to the invention.

FIG. 21B illustrates the individual wrapping body in the sixth embodiment according to the invention.

FIG. 22 is a front view of an inside wrapping body in a seventh embodiment according to the invention.

FIG. 23A illustrates an individual wrapping body in the seventh embodiment according to the invention.

FIG. 23B illustrates the individual wrapping body in the seventh embodiment according to the invention.

FIG. 23C illustrates the individual wrapping body in the seventh embodiment according to the invention.

FIG. 24 is a front view of an inside wrapping body in an eighth embodiment according to the invention.

FIG. 25 is a front view of an inside wrapping body in a ninth embodiment according to the invention.

FIG. 26 is a front view of an inside wrapping body in a tenth embodiment according to the invention.

FIG. 27 is a front view of an individual wrapping container in an eleventh embodiment according to the invention.

FIG. 28 is a cross-sectional view of the individual wrapping container in the eleventh embodiment according to the invention.

FIG. 29A is a front view of the individual wrapping container in the eleventh embodiment according to the invention.

FIG. 29B is a front view of the individual wrapping container in the eleventh embodiment according to the invention.

FIG. 29C is a front view of the individual wrapping container in the eleventh embodiment according to the invention.

FIG. 29D is a front view of the individual wrapping container in the eleventh embodiment according to the invention.

FIG. 29E is a front view of the individual wrapping container in the eleventh embodiment according to the invention.

FIG. 30 is a front view of the individual wrapping container in the eleventh embodiment according to the invention.

FIG. 31A is a front view of the individual wrapping container in the eleventh embodiment according to the invention.

FIG. 31B is a front view of the individual wrapping container in the eleventh embodiment according to the invention.

FIG. 31C is a front view of the individual wrapping container in the eleventh embodiment according to the invention.

FIG. 31D is a front view of the individual wrapping container in the eleventh embodiment according to the invention.

FIG. 32 is a front view of the individual wrapping container in a twelfth embodiment according to the invention.

FIG. 33A is a front view of an individual wrapping body in the twelfth embodiment according to the invention.

FIG. 33B is a front view of the individual wrapping body in the twelfth embodiment according to the invention.

FIG. 33C is a front view of the individual wrapping body in the twelfth embodiment according to the invention.

FIG. 33D is a front view of the individual wrapping body in the twelfth embodiment according to the invention.

FIG. 34 is a perspective view of an outer wrapping container in a thirteenth embodiment according to the invention.

FIG. 35A is a perspective view of the outer wrapping container in the thirteenth embodiment according to the invention.

FIG. 35B is a perspective view of the outer wrapping container in the thirteenth embodiment according to the invention.

FIG. 35C is a perspective view of the outer wrapping container in the thirteenth embodiment according to the invention.

FIG. 36 is a perspective view of the outer wrapping container in the thirteenth embodiment according to the invention.

FIG. 37 is a perspective view of an intermediate wrapping container in the thirteenth embodiment according to the invention.

FIG. 38 is a perspective view of the intermediate wrapping container in the thirteenth embodiment according to the invention.

FIG. 39A is a perspective view illustrating steps until taking a interlabial pad out of the outer wrapping wrapping container in the thirteenth embodiment according to the invention.

FIG. 39B is a perspective view illustrating steps until taking a interlabial pad out of the outer wrapping wrapping container in the thirteenth embodiment according to the invention.

FIG. 39C is a perspective view illustrating steps until taking a interlabial pad out of the outer wrapping wrapping container in the thirteenth embodiment according to the invention.

FIG. 39D is a perspective view illustrating steps until taking a interlabial pad out of the outer wrapping wrapping container in the thirteenth embodiment according to the invention.

FIG. 40 is a perspective view of an out wrapping container in a fourteenth embodiment according to the invention.

FIG. 41 is a perspective view of the outer wrapping container in the fourteenth embodiment according to the invention.

FIG. 42 is a front view of an individual wrapping body of a related art.

FIG. 43A illustrates an outer wrapping container of a related art.

FIG. 43B illustrates the outer wrapping container of a related art.

FIG. 43C illustrates the outer wrapping container of a related art.

FIG. 43D illustrates the outer wrapping container of a related art.

### DESCRIPTION OF THE PREFERRED EMBODIMENT

Preferred embodiments of the present invention are hereinafter described with reference to the appended drawings. In the description of the following embodiments, similar reference numerals are given to similar components, and explanation for similar components is omitted or briefly made. A. Individual Wrapping Container for Protecting Peripheral portion of Interlabial pad

### First Embodiment

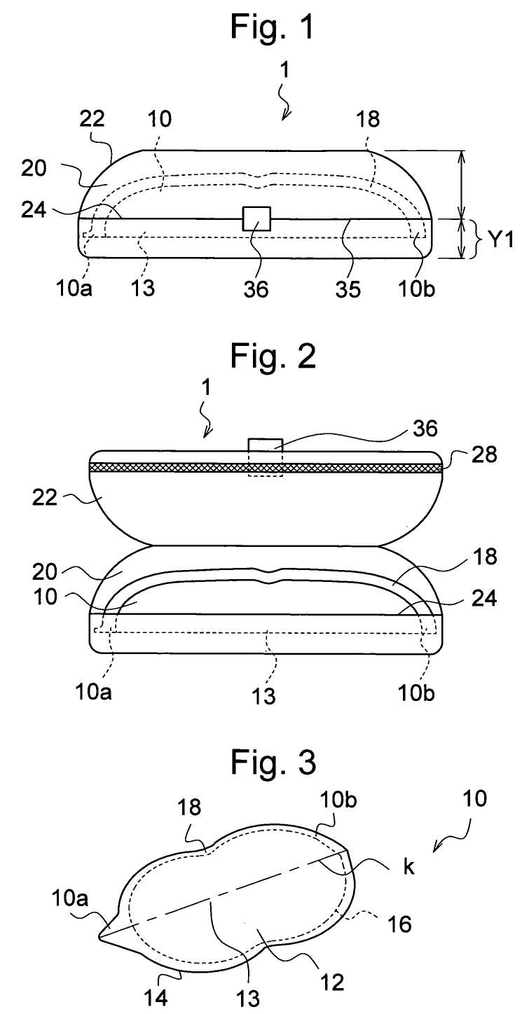

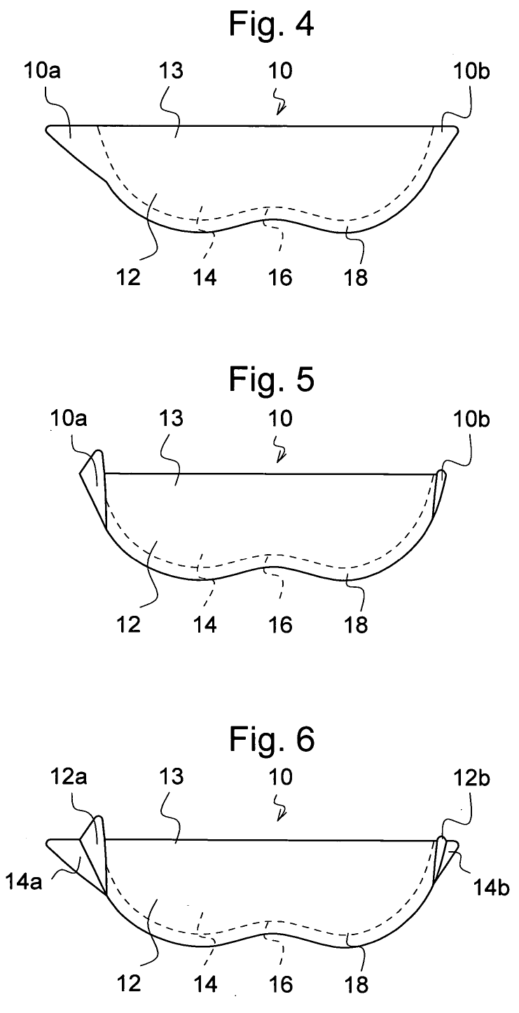

FIG. 1 is a front view of an individual wrapping body 1 in a first embodiment according to the present invention. FIG. 2 illustrates a condition where an individual wrapping container 20 of the individual wrapping body 1 in the first embodiment of the invention is opened. FIG. 3 is a perspective view of an interlabial pad 10 in the first embodiment of the invention. FIG. 4 is a front view illustrating the interlabial pad 10 in the first embodiment of the invention folded double along a center line k in a longitudinal direction.

As illustrated in FIG. 1, the individual wrapping body 1 includes the interlabial pad 10 and the individual wrapping container 20 in which the interlabial pad 10 is packaged.

The interlabial pad 10 is attached between labia of a wearer. As illustrated in FIG. 3, the interlabial pad 10 includes: a surface side sheet 12 as a covering body which is made from a liquid-permeable material having a feature which would allow the liquid to be transmitted; a back side sheet 14 as a covering body which is made from a non-liquid-permeable material having a feature which would substantially not allow the liquid to be transmitted; and an absorption member 16 interposed between the surface side sheet 12 and the back side sheet 14. The interlabial pad 10 also includes a peripheral portion 18 forming a contour of the interlabial pad 10. A front end 10a and a rear end 10b are provided at end portions of the peripheral portion 18 in the longitudinal direction. As illustrated in FIG. 3, the interlabial pad 10 is folded double along the center line k in the longitudinal direction such that the two parts of the back side sheet 14 facing to each other at the time of attachment, thereby providing a shape illustrated in FIG. 4. The interlabial pad 10 additionally includes a vestibular floor contact region 13 for contacting the vestibular floor of the wearer during attachment.

The surface side sheet 12 and the back side sheet 14 covering the absorption member 16 have a surface side sheet front end 12a and a back side sheet front end 14a positioned on the pubis side during attachment, and a surface side sheet rear end 12b and a back side sheet rear end 14b positioned on the buttocks side during attachment, respectively, in the above-described vestibular floor contact region 13. In this embodiment, the surface side sheet front end 12a and the back side sheet front end 14a constitute the front end 10a of the peripheral portion 18 of the interlabial pad 10, and the surface side sheet rear end 12b and the back side sheet rear end 14b constitute the rear end 10b of the peripheral portion 18.

As illustrated in FIGS. 1 and 2, the individual wrapping container 20 is formed by a sheet body 22 which covers and accommodates the entire interlabial pad 10. The individual wrapping container 20 has an unwrapped opening 24. The sheet body 22 partially overlies itself in the vicinity of the unwrapped opening 24, and the overlapped regions of the sheet body 22 are bonded with each other in such a manner as to be openable and separable.

The above-described individual wrapping container 20 illustrated in FIG. 1 includes a protective region Y1 having higher rigidity than that of the peripheral portion 18 of the interlabial pad 10. The protective region Y1 is so sized as to cover a portion corresponding to the vestibular floor contact region 13, i.e., 5 to 70% of the width of the interlabial pad 10 in the condition where the interlabial pad 10 is packaged in the individual wrapping container 20, and continuously extends in the longitudinal direction. An embossed seal 28 for joining the overlapped regions of the sheet body 22 constituting the individual wrapping container 20 is provided on an end side of the sheet body 22. The embossed seal 28 extends in the longitudinal direction of the individual container 20, increasing the rigidity of the protective region Y1. Additionally, adhesive is applied between the overlapped regions of the sheet body 22 continuously in the longitudinal direction so as to enhance sealing of the individual wrapping container 20. This adhesive further increases the rigidity of the individual wrapping container 20. Thus, even if external pressure is applied to the individual wrapping body 1 while carrying it, the shape of the vestibular floor contact region 13 is maintained. Additionally, the individual wrapping body 1 is formed such that the interlabial pad 10 is completely sealed in the individual wrapping container 20 with air contained therein, thereby preventing damage to the interlabial pad 10 in the individual wrapping body 1 even in a region other than the peripheral portion 18 even when the individual wrapping body 1 is dropped.

The embossed seal 28 for joining the overlapped regions of the sheet body 22 constituting the individual wrapping container 20 may be provided throughout the peripheral portion of the individual wrapping container 20. Additionally, a closing tape 36 having adhesion may be provided at an unwrapped portion 35 formed by the overlapped regions of the sheet body 22 so as to make it easy to unwrap.

In the present invention, it is possible to prevent warp of the front end 10a which is easy to contact the clitoris of the wearer and the rear end 10b which is easy to contact the anus of the wearer during attachment in the peripheral portion 18 of the interlabial pad 10 as illustrated in FIG. 5 by providing the protective region Y1 in the individual packing container 20. Thus, the clitoris and anus of the wearer are not damaged at the time of attachment of the interlabial pad 10. It is also possible to prevent warp of the surface side sheet front end 12a and the surface side sheet rear end 12b and resultant separation thereof from the back side sheet front end 14a and the back side sheet rear end 14b, respectively, and also exposure of the absorption member 16 in the peripheral portion 18 as illustrated in FIG. 6. Thus, fibers of the absorption member 16 do not remain on the inner walls of the labia due to contact therebetween during attachment.

The protective region Y1 of the individual wrapping container 20 may be provided for protecting either the entire peripheral portion 18 of the interlabial pad 10 or only the vestibular floor contact region 13. Alternatively, the protective region Y1 of the individual wrapping container 20 may be provided for protecting only the front end 10a (the surface side sheet front end 12a and the back side sheet front end 14a) or the rear end 10b (the surface side sheet rear end 12b and the back side sheet rear end 14b).

The buckling strength of the protective region Y1 of the individual wrapping container 20 is preferably in a range from 100 mN to 2,000 mN. By establishing the strength in this range, warp of the peripheral portion 18 while carrying the individual wrapping body 1 can be prevented in the wrapping process. The buckling strength of the peripheral portion 18 of the interlabial pad 10 is preferably in a range from 50 mN to 400 mN.

### Second Embodiment

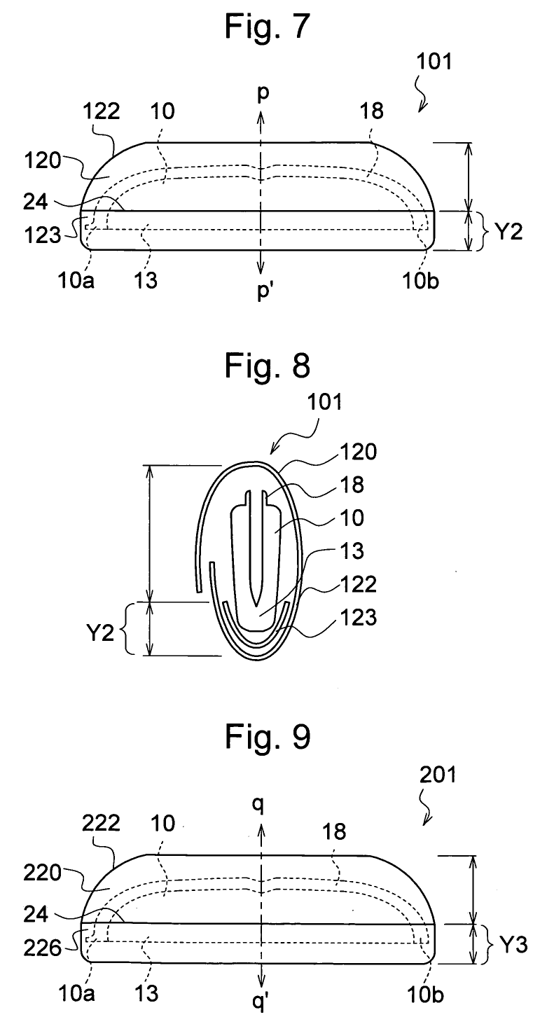

FIG. 7 is a front view of an individual wrapping body 101 in a second embodiment according to the present invention. FIG. 8 is a cross-sectional view of the individual wrapping body 101 taken along a line p-p′ in FIG. 7.

The individual wrapping body 101 in this embodiment is different from the individual wrapping body 1 in the first embodiment in that more sheet bodies constituting an individual wrapping container 120 are provided in this embodiment than in the first embodiment. That is, a protective region Y2 is formed by additionally disposing a small sheet body 123 between a sheet body 122 and the interlabial pad 10 as illustrated in FIGS. 7 and 8. Since the small sheet body 123 is positioned in the protective region Y2 which includes a portion corresponding to the vestibular floor contact region 13, the rigidity of that portion is increased by laminating the sheet body 122 and the small sheet body 123.

### Third Embodiment

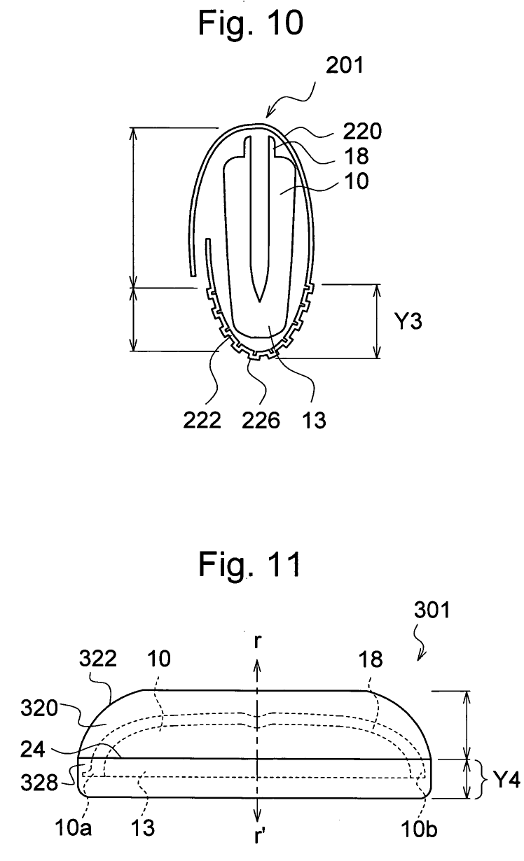

FIG. 9 is a front view of an individual wrapping body 201 in a third embodiment according to the present invention. FIG. 10 is a cross-sectional view of the individual wrapping body 201 taken along a line q-q′ in FIG. 9.

The individual wrapping body 201 in this embodiment is different from the individual wrapping body 1 in the first embodiment in that a protective region Y3 is provided by embossing an embossed pattern 226 on a sheet body 222 constituting an individual wrapping container 220 in an area contacting the vestibular floor contact region 13 of the interlabial pad 10 as illustrated in FIGS. 9 and 10. The protective region Y3 in which the embossed pattern 226 is provided has high rigidity and can be easily formed at an appropriate position by embossing. Moreover, since an additional component is not required for forming the protective region Y3, the material cost can be reduced.

The embossed pattern 226 may be provided on the protective region Y3 of the sheet body 222, may be provided on an entire area of the sheet body 222 contacting the peripheral portion 18 of the interlabial pad 10, or may be formed on the entire sheet body 222.

### Fourth Embodiment

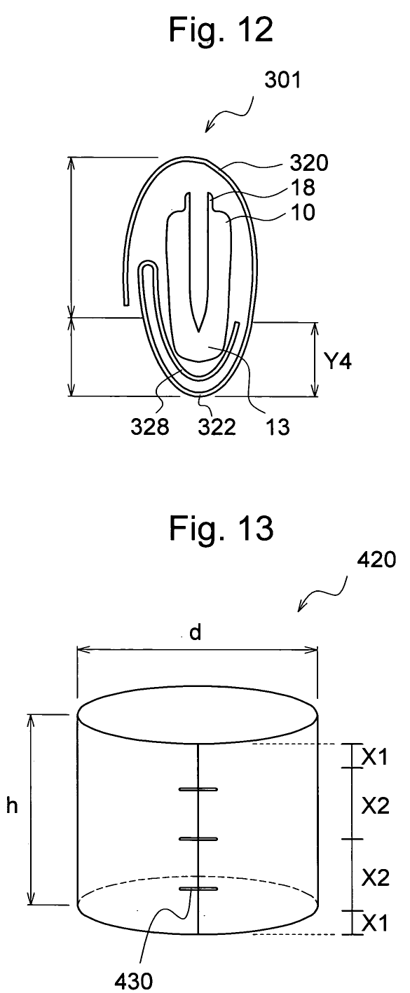

FIG. 11 is a front view of an individual wrapping body 301 in a fourth embodiment according to the present invention. FIG. 12 is a cross-sectional view of the individual wrapping body 301 taken along a line r-r′ in FIG. 11.

The individual wrapping body 301 in this embodiment is different from the individual wrapping body 1 in the first embodiment in that an individual wrapping container 320 includes a protective region Y4 which is formed by a folded portion 328 produced by folding a part of a sheet body 322. This structure not only increases the buckling strength of the individual wrapping container 320 but also allows the protective region Y4 to have high rigidity and be formed at an appropriate position without requiring an additional component. Since an additional component which is used in the second embodiment is not required for forming the protective region Y4 in this embodiment, the material cost can be reduced.

Measuring Buckling Strength of Individual Wrapping Container

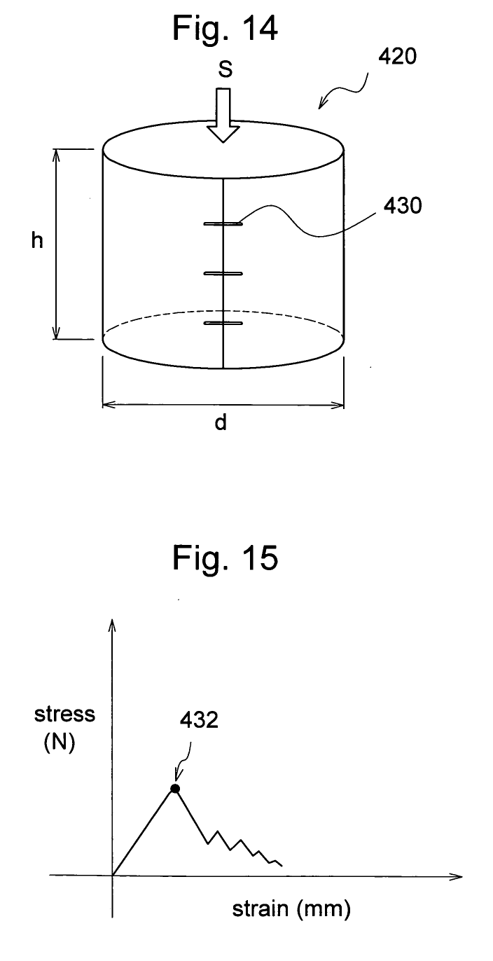

FIGS. 13 and 14 show a method for measuring buckling strength of each material constituting an individual wrapping container of the invention employing a method for measuring coefficient of static friction. The buckling strength is measured using an Instron all-purpose material testing machine (manufactured by Instron Japan Co. Ltd.). To start with, a cylindrical individual wrapping container 420 having a diameter “d” of 45 mm and a height “h” of 40 mm is formed as illustrated in FIG. 13. Then, the cylindrical individual wrapping container 420 is joined by staples 430 of a stapler at intervals of X1 and X2. In the measurement of the present invention, the values of X1 and X2 for joining are 5 mm and 15 mm, respectively. Subsequently, the buckling strength of the individual wrapping container 420 is measured setting a compression speed “S” of the testing machine at 30 mm/min. as illustrated in FIG. 14. When the protective region has four layers (two layers on one side with the interlabial pad disposed in the middle) with an additional small sheet provided as in the example of FIG. 8, a two-layered cylindrical specimen is prepared for the measurement.

FIG. 15 shows the relationship between stress applied to the individual wrapping container 420 and distortion. In this experiment, the buckling strength is determined according to an average value from five stress measurements at a first yield point 432 at the time of 80% compression of a specimen. The buckling strength at the peripheral portion (the surface side sheet and the back side sheet) is measured by the same method as in the measurement of the individual wrapping container 420.

Component Material

Individual Wrapping Container

Examples of material for the individual wrapping container involve polyethylene, polypropylene, polyethylene terephthalate, polyvinyl alcohol, polylactate, polybutyl succinate, nonwoven fabrics, paper, and laminated material thereof each of which is 15 to 60 μm in thickness. Alternatively, a breathable film obtained by drawing a composite sheet of a resin and an inorganic filler may be used. More specifically, a film formed by mixing low density polyethylene (LDPE) resin and high density polyethylene (HDPE) resin and adjusting the basis weight per unit are in a range from 15 to 35 g/m² may be used. Other examples include an individual wrapping container material having a film formed from polyethylene, polypropylene or others, or a material having a melt-blown surface formed by spraying melt-blown on nonwoven fabrics or paper on the inside surface of the individual wrapping container. Since the melt-blown is a collection of extrafine fibers and has a large surface area, a coefficient of static friction in an adequate range can be obtained. Additionally, the individual wrapping container preferably blocks off the color of the body fluid absorbed by the interlabial pad. For this purpose, pigment may be mixed in a range from 0.2% to 10%, or ink and the like may be printed. Examples of material for the individual wrapping container having breathability in a range from 0.040 Kpa·s/m to 1,000 KPa·s/m involve a breathable film obtained by drawing a composite sheet of a resin and an inorganic filler or by providing connected pores having been subjected to pore-opening treatment, and polyethylene, polypropylene, polyethylene terephthalate, polyvinyl alcohol, polylactate, polybutyl succinate, nonwoven fabrics, paper, and laminated material thereof having connected pores, each of which is 15 to 60 μm in thickness. More specifically, so-called SMS nonwoven fabrics including 6-10 gsm/5-20 gsm/6-10 gsm of spunbond/melt-blown/spunbond may be used. Since the melt-blown is a collection of extrafine fibers and has high barrier property due to its high density, adequate breathability can be obtained by adjusting the basis weight per unit area or other conditions of the melt-blown layer.

Surface Side Sheet of Interlabial Pad

Material which has water-permeability and liquid-hydrophilic property and gives no stimulation to the skin is used for the surface side sheet of the interlabial pad disposed on the body side. Examples of such material include pulp, chemical pulp, rayon, cotton, acetate, synthetic fibers, rayon or acetate which are physically embossed, chemical pulp bridged by a bridging material and crimped, each of which is used alone or combined. The sheet is manufactured by methods such as spunbonding, point-bonding, air-through, melt-blown, spunlacing and papermaking. Among these methods, spunlacing is the most preferable which is capable of intergangling fibers by hydroentanglement and of removing oil adhering to fibers by the water flow, since the outside surface of the interlabial pad preferably contains no oil, lubricant and the like which may increase slippage between the outside surface of the pad and the inside surface of the individual wrapping container.

Back Side Sheet of Interlabial Pad

The liquid-impermeable back side sheet included in the interlabial pad is made from a water-impermeable material which is capable of preventing leakage of menstrual blood retained by the absorption member out of the interlabial pad. Further, the back side sheet is made from material having moisture permeability, thereby reducing moisture and uncomfortableness during attachment. Examples of the back side sheet as described involve polyethylene, polypropylene, polyethylene terephthalate, polyvinyl alcohol, polylactate, polybutyl succinate, nonwoven fabrics, paper, and laminated material thereof, each of which is 15 to 60 μm in thickness. Alternatively, a breathable film obtained by drawing a composite sheet of a resin and an inorganic filler may be employed. More specifically, a film prepared using a low density polyethylene (LDPE) resin as a main component and adjusted to have a basis weight per unit area in a range from 15 to 35 g m² may be used. Other examples include a back side sheet having a film formed from polyethylene, polypropylene or others, or a melt-blown surface formed by spraying melt-blown on nonwoven fabrics or paper on the outside surface of the interlabial pad. Since the melt-blown is a collection of extrafine fibers and has a large surface area, a coefficient of static friction in an adequate range can be obtained. B. Individual Wrapping Container for Preventing Contamination of Interlabial pad

### Fifth Embodiment

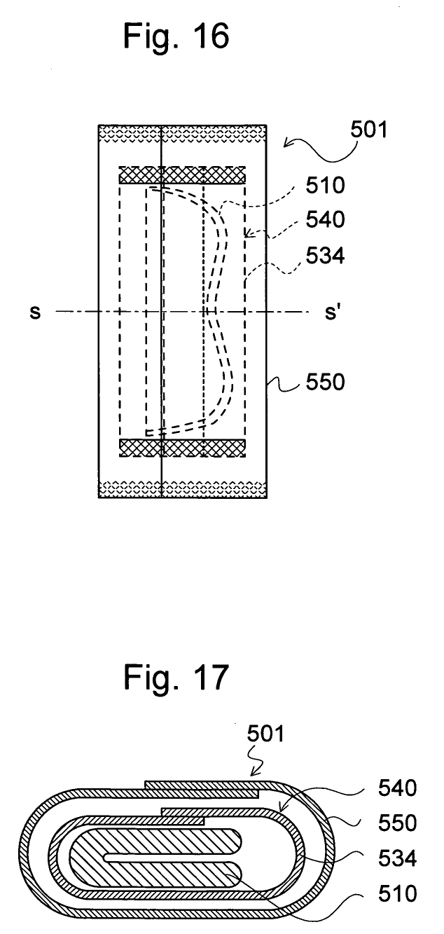

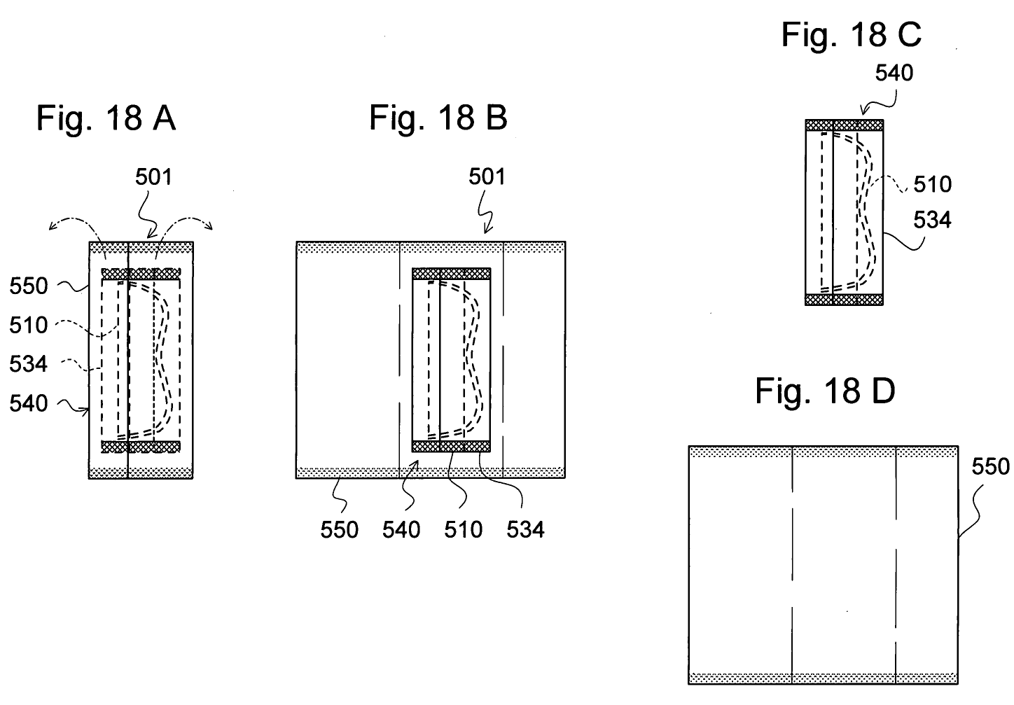

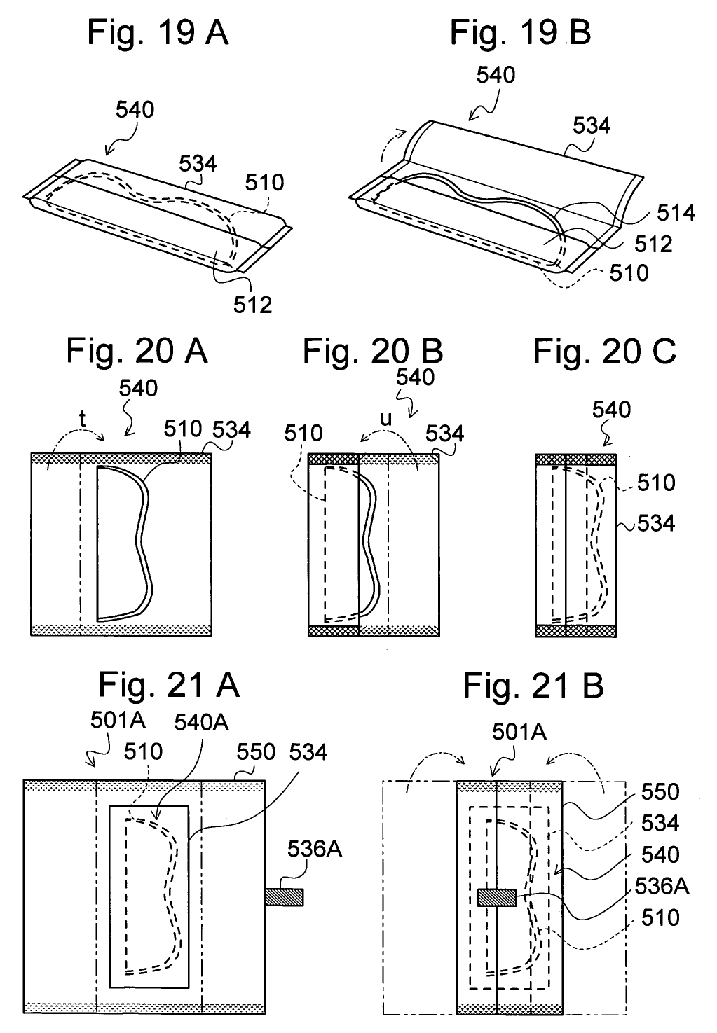

FIG. 16 is a front view of an individual wrapping body 501 in a fifth embodiment according to the present invention. FIG. 17 is a cross-sectional view of the individual wrapping body 501 taken along a line s-s′ in FIG. 16. FIG. 18A is a front view of the individual wrapping body 501 in the fifth embodiment of the invention. FIG. 18B illustrates the unfolded individual wrapping body 501 in the fifth embodiment of the invention. FIG. 18C is a front view of an inner wrapping body 540 in the fifth embodiment of the invention. FIG. 18D is a front view of a discard sheet 550 in the fifth embodiment of the invention. FIG. 19A is a perspective view of the inner wrapping body 540 in the fifth embodiment of the invention. FIG. 19B is a perspective view illustrating a condition where a protective sheet 534 of the inside wrapping body 540 in the fifth embodiment of the invention is partially unfolded. FIG. 20A illustrates a condition where the protective sheet 534 of the individual wrapping body 501 is unfolded in the fifth embodiment of the invention. FIG. 20B is a perspective view illustrating a condition where the protective sheet 534 of the individual wrapping body 501 is partially unfolded in the fifth embodiment of the invention. FIG. 20C is a front view of the inner wrapping body 540 in the fifth embodiment of the invention.

In this embodiment, the individual wrapping body 501 includes the inside wrapping body 540 and the discard sheet 550 for accommodating the inner wrapping body 540 as illustrated in FIG. 16. The inner wrapping body 540 includes an interlabial pad 510 and the protective sheet 534 for directly wrapping the interlabial pad 510 before use.

In this embodiment, both sides of the protective sheet 534 in the width direction are folded in such a manner as to surround the interlabial pad 510, thereby forming the inner wrapping body 540 as illustrated in FIG. 17. Also, both sides of the discard sheet 550 in the width direction are folded in such a manner as to surround the inner wrapping body 540, thereby forming the individual wrapping body 501 in which the inner wrapping body 540 is covered by the discard sheet 550. In use of the individual wrapping body 501 illustrated in FIG. 18A, the discard sheet 550 is unfolded as illustrated in FIG. 18B, and then the discard sheet 550 and the inner wrapping body 540 are separated as illustrated in FIGS. 18C and 18D. Thus, while the used interlabial pad is being wrapped in the discard sheet 550 for discarding the used pad, the interlabial pad 510 before use can be kept packaged in the protective sheet 534 so as to prevent contamination of the interlabial pad 510 before use.

When an unwrapped opening of the protective sheet 534 is opened in this embodiment, a back side sheet 514 of the interlabial pad 510 or a holding portion to be held at the time of attachment appear as illustrated in FIGS. 19A and 19B. Thus, the wearer can hold the back side sheet 514 or the holding portion to be held at the time of attachment and take out the interlabial pad 510.

Next, steps for wrapping the interlabial pad 510 before use in the protective sheet 534 in this embodiment are shown in FIGS. 20A, 20B and 20C. First, one side of the protective sheet 534 in a horizontal axis direction is folded such that a back side sheet 514 of the interlabial pad 510 before use or the side on which the holding portion to be held at the time of attachment is not formed can be wrapped (hereinafter referred to as “first fold t”) as illustrated in FIG. 20A. Then, the other side is folded (hereinafter referred to as “second fold u”) and the overlapped portions of the protective sheet 534 are closed as illustrated in FIG. 20B. Subsequently, the overlapped ends of the protective sheet 534 which ends extend in a vertical axis direction are re-openably bonded to each other by heat-embossing pressure or by the tangle between male and female portions of the embossment as illustrated in FIG. 20C.

The dimension of the first fold “t” is determined such that more than half of the entire length of the interlabial pad 510 before use in a lateral direction is covered, preferably 5 to 60 mm. The dimension of the second fold “u” is determined such that the second fold “u” is overlapped on the outside surface of the first fold “t,” preferably 10 to 60 mm. The dimension of the overlapped portions of the first fold “t” and the second fold “u” is preferably 5 to 20 mm. The overlapped portions are closed by a known technique such as closing tape, hot melt adhesive, heat sealing and mating embossment so as to prevent entrance of dirt, dust etc. More specifically, the overlapped portions are closed by applying hot melt adhesive having a basis weight per unit area in a range from 5 to 10 g/m² and a width from 1 to 3 mm on the inside surface of the protective sheet 534 in an area of 2 to 10 mm from the end of the second fold “u” in the overlapping direction. The overlapped ends of the protective sheet 534 in the vertical axis direction are heat embossed at the fusion temperature of the protective sheet 534 to be re-separably bonded to each other. More specifically, the protective sheet 534 is made from polyethylene film having a basis weight per unit area of 25 g/m² which is heat sealed using an embossing pattern roll at a temperature from 70 to 120° C. for the upper axis roll and at a temperature from 90 to 140° C. for the lower axis roll. The embossing pattern roll used herein has embossments each having an area of 0.7 mm² and disposed close to each other with a clearance of 1 mm between adjoining embossments with three rows of embossment parts at an angle of 45 degrees.

The term “apparent” refers to the minimum “length” of a distance between two points. This is a term definition just to make sure, since there is a case when the actual “length” refers to a “way” between two points along concave and convex portions (i.e., a distance between two points in a flat condition where concave and convex portions are spread out) in view of the production process.

### Sixth Embodiment

FIG. 21A illustrates an individual wrapping body 501A in an unfolded condition in a sixth embodiment of the present invention. FIG. 21B shows the individual wrapping body 501A in the sixth embodiment of the invention.

As illustrated in FIGS. 21A and 21B, the individual wrapping body 501A in this embodiment is different from the individual wrapping body 501 in the above-described fifth embodiment in that the discard sheet 550 has a closing tape 536A. FIGS. 21A and 21B show the steps for wrapping an inner wrapping body 540A in the discard sheet 550 in this embodiment. First, the inner wrapping body 540A is disposed substantially at the center of the discard sheet 550, and both sides of the discard sheet 550 in the horizontal axis direction are folded such that the inner wrapping body 540A is surrounded by those sides. Subsequently, the overlapped ends of the discard sheet 550 in the vertical axis direction are re-separably bonded to each other, and are then closed by the closing tape 536A attached to the discard sheet 550. This structure limits the movement of the inner wrapping body 540A packaged in the discard sheet 550, and prevents the inner wrapping body 540A from being dropped out of the discard sheet 550. When the used interlabial pad is wrapped in the discard sheet 550 and discarded, the discard sheet 550 in which the used interlabial pad is accommodated can be closed by the use of the adhesion of the closing tape 536A. It is also possible to prevent contamination of the wearer's hand which may be caused when the discard sheet 550 in which the used interlabial pad 510 is wrapped is re-opened before discarding the pad into a sanitary container, thereby avoiding insanitation.

The length of the discard sheet 550 is equal to or larger than the length of the inner wrapping body 540A, specifically 80 to 300 mm in the vertical axis direction and 50 to 400 mm in the horizontal axis direction. The method for bonding the overlapped ends of the discard sheet 550 which ends extend in the vertical axis direction is the same as that for bonding the overlapped ends of the above-described protective sheet 534 which ends extend in the vertical axis direction. The method for closing the overlapped portions of the discard sheet 550 is not limited to the use of the closing tape 536A, but may be by a known technique such as heat sealing, mating embossment, and applying a small amount of adhesive along the overlapping direction.

The discard sheet 550 is not limited to the three-fold shape as illustrated in FIGS. 21A and 21B. It is more preferable that the end of the discard sheet 550 not having the closing tape 536A is folded inside to provide a sufficient size for wrapping the used interlabial pad when the discard sheet 550 is opened. Since the used interlabial pad is wrapped in the discard sheet 550 with sufficient margins, accidental contamination of the wearer's hand by the menstrual blood adhering to the used interlabial pad is avoided. Further, the discard sheet 550 may be used as a sheet for covering the wearer's fingers at the time of removal of the used pad from the labia so as to prevent contamination of the fingers by the menstrual blood.

### Seventh Embodiment

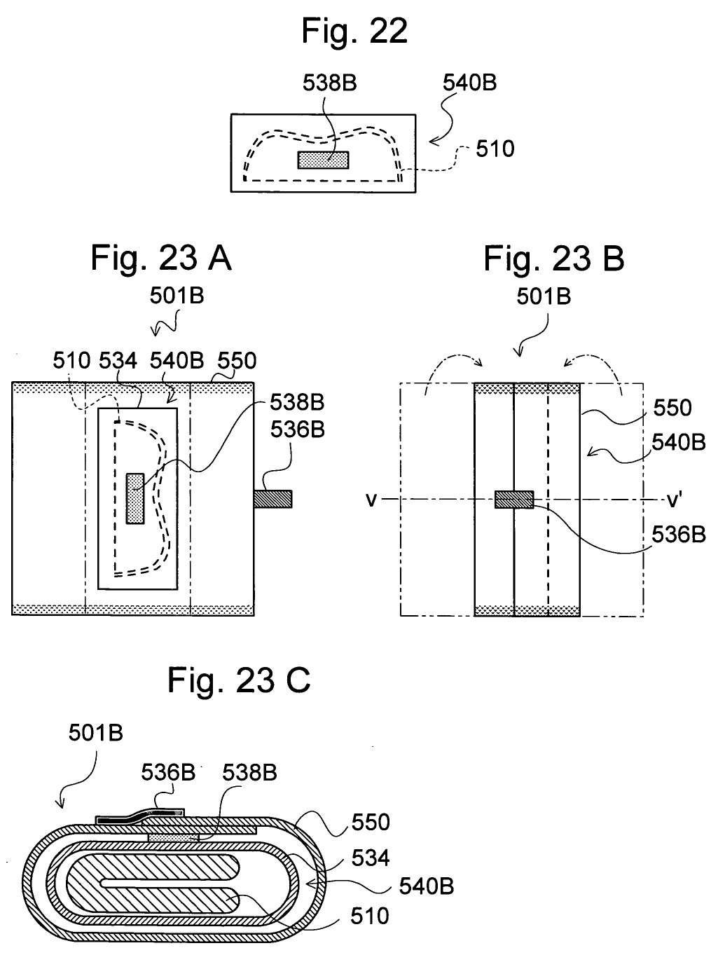

FIG. 22 is a front view of an inner wrapping body 540B in a seventh embodiment of the present invention. FIG. 23A illustrates an individual wrapping body 501B in an unfolded condition in the seventh embodiment of the invention. FIG. 23B is a front view of the individual wrapping body 501B in the seventh embodiment of the invention. FIG. 23C is a cross-sectional view of the individual wrapping body 510B taken along a line v-v′ in FIG. 23B.

The individual wrapping body 501B in this embodiment is different from the individual wrapping body 501 in the above-described fifth embodiment in that the inside wrapping body 540B has adhesive 538B as illustrated in FIG. 22 and that the discard sheet 550 has a closing tape 536B as illustrated in FIGS. 23A and 23B. The inner wrapping body 540B includes the adhesive 538B at least in one position which is located out of the unwrapped opening of the outside surface of the protective sheet 534 covering the interlabial pad 510. The inner wrapping body 540B is packaged in the discard sheet 550 as illustrated in FIGS. 23A and 23B. One side of the discard sheet 550 in the longitudinal direction is so folded as to cover the adhesive 538B provided on the inner wrapping body 540B, and is closed by the closing tape 536B. The method for closing the end in the horizontal axis direction and the overlapped portions is the same as that in the above-described sixth embodiment.

The adhesive 538B provided on the outside surface of the protective sheet 534 covering the interlabial pad 510 is required to be covered only by a single surface of the discard sheet 550 without extending over the overlapped portions of the discard sheet 550. More preferably, the individual wrapping body 501B is structured such that the side of the discard sheet 550 folded first is opposed to the adhesive 538B. Since the inner wrapping body 540B is kept attached to the discard sheet 550 until the discard sheet 550 is sufficiently spread out, accidental dropping of the interlabial pad 510 from the inside wrapping body 540B is prevented. After opening the discard sheet 550, the protective sheet 534 covering the interlabial pad 510 is taken out with the adhesive 538B attached to the outside surface of the protective sheet 534. Thus, the inner wrapping body 540B can be fixed to a wall, a paper holder or other place by use of the adhesion of the adhesive 538B. Accordingly, the inner wrapping body 540B can be temporarily placed, thereby preventing contamination of the surface of the interlabial pad 510 resulting from slipping of the pad 510 from the protective sheet 534 which may be caused at the time of discarding the used interlabial pad while holding the inside wrapping body 540B by hand.

### Eighth Embodiment

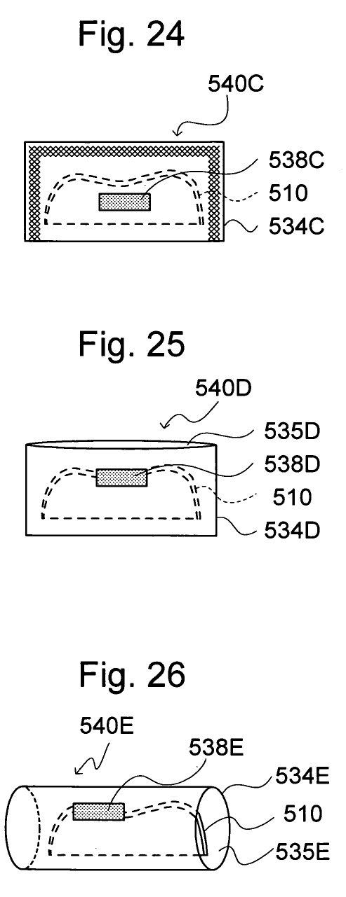

FIG. 24 is a front view of an inner wrapping body 540C in an eighth embodiment of the present invention.

The inner wrapping body 540C in this embodiment is different from the inner wrapping body 540 in the above-described fifth embodiment in that the inner wrapping body 540C is completely sealed by sealing three sides of a protective sheet 534C other than the folded portion thereof as illustrated in FIG. 24. Since the interlabial pad 510 is completely sealed within the protective sheet 534C, dropping of the pad 510 from the protective sheet 534C is prevented. Additionally, it is possible to temporarily place the interlabial pad 510 covered with the protective sheet 534C by attaching the protective sheet 534C to a wall, a paper holder or other place in either a vertical or horizontal direction in accordance with a space in a lavatory to which the protective sheet 534C is attachable. Moreover, not only is the interlabial pad 510 completely sealed within the protective sheet 534C, but also adhesive 538C is provided at least at one position which is located other than the unwrapped opening of the outside surface of the protecting sheet 534C covering the interlabial pad 510. Thus, contamination of the surface of the interlabial pad 510 resulting from slipping of the pad 510 from the protective sheet 534C is avoided. Furthermore, after wrapping the used interlabial pad in the discard sheet and discarding it, the inner wrapping body 540C is removed from the place to which it has been attached and is opened, so that the wearer can attach the interlabial pad 510 in a clean condition.

### Ninth Embodiment

FIG. 25 is a front view of an inner wrapping body 540D in a ninth embodiment of the present invention.

The inner wrapping body 540D in this embodiment is different from the innner wrapping body 540 in the above-described fifth embodiment in that the inner wrapping body 540D is packaged such that the back side sheet or the holding portion of the interlabial pad 510 faces to an unwrapped opening 535D of a protective sheet 534D as illustrated in FIG. 25. This structure allows the protective sheet 534D to be fixed to a wall, a paper holder or other place in the lavatory in such a manner that the unwrapped opening 535D is always located at an upper position in the vertical direction without dropping of the interlabial pad 510 from the protective sheet 534D which may be caused by the expansion of the unwrapped opening 535D due to the weight of the interlabial pad 510. Thus, the protective sheet 534D can be temporality placed without slipping of the interlabial pad 510 from the protective sheet 534D and resultant contamination of the surface of the pad 510. Moreover, since the unwrapped opening from which the interlabial pad 510 is taken out is open in advance in this embodiment, the steps from taking out the interlabial pad 510 before use till attaching the pad 510 are shortened compared with the above-described eighth embodiment in which the interlabial pad 510 is completely sealed in the protective sheet 534C. Thus, the attachment of the pad 510 is facilitated without contaminating the clothes and the body of the wearer or the inside of the lavatory by the menstrual blood flowing out of the wearer's vaginal opening.

### Tenth Embodiment

FIG. 26 is a front view of an inner wrapping body 540E in a tenth embodiment of the present invention.

The inner wrapping body 540E in this embodiment is different from the inner wrapping body 540 in the fifth embodiment in that the interlabial pad 510 is accommodated in a protective sheet 534E such that an unwrapped opening 535E of the protective sheet 534E is disposed perpendicularly to the longitudinal direction of the interlabial pad 510 as illustrated in FIG. 26. The width of applied adhesive 538E is preferably more than one third of the length of the protective sheet 534E parallel to the longitudinal direction of the interlabial pad 510, and is preferably located substantially at the center of the length perpendicular to the side of the protective sheet 534E which side includes the unwrapped opening 535E. The adhesive 538E thus provided prevents dropping of the interlabial pad 510 from the tilted protective sheet 534E when the protective sheet 534E is attached to a wall or a paper holder such that the unwrapped opening 535E is horizontally disposed.

### Eleventh Embodiment

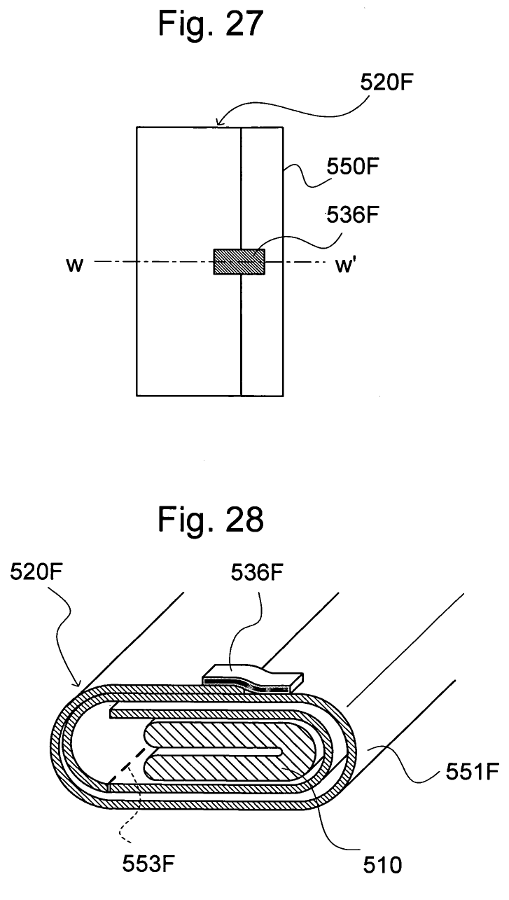

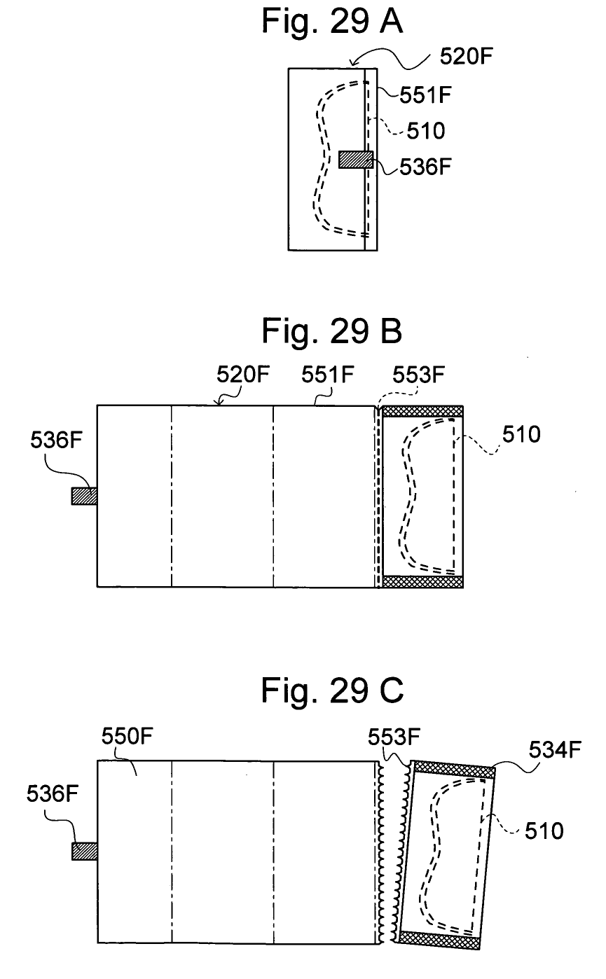

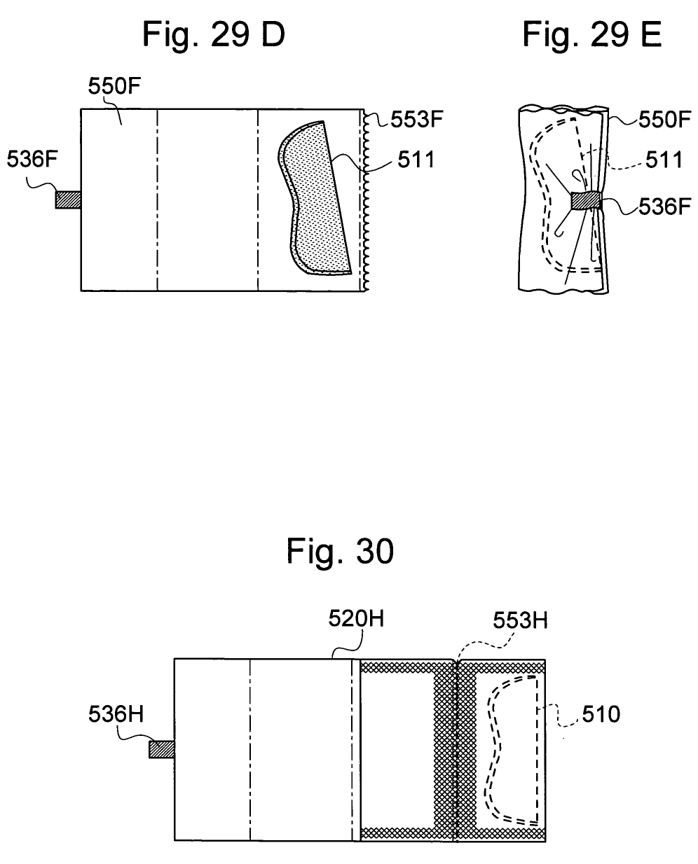

FIG. 27 is a front view of an individual wrapping container 520F in which the interlabial pad 510 is packaged in an eleventh embodiment of the present invention. FIG. 28 is a cross-sectional view of the individual wrapping container 520F taken along a line w-w′ in FIG. 27. FIG. 29A is a front view of the individual wrapping container 520F in which the interlabial pad 510 is packaged in the eleventh embodiment of the invention. FIG. 29B illustrates the unfolded individual wrapping container 520F in which the interlabial pad 510 is packaged in the eleventh embodiment of the invention. FIG. 29C illustrates the individual wrapping container 520F in which the interlabial pad 510 is packaged in the eleventh embodiment of the invention, with the individual wrapping container 520F unfolded and broken along a breaking line 553F. FIG. 29D illustrates the individual wrapping container 520F in which the interlabial pad 510 is packaged in the eleventh embodiment of the invention, with the individual wrapping container 520G unfolded and broken along a breaking line 553F. FIG. 29E illustrates a condition where the used interlabial pad 510 is packaged in a wrapping sheet 551F in the eleventh embodiment of the invention.

The individual wrapping container 520F in this embodiment is different from the example of the fifth embodiment in that the individual wrapping container 520F is formed by a successive wrapping sheet 551F as illustrated in FIGS. 27 and 28. The interlabial pad 510 is disposed at one end side of the wrapping sheet 551F in the longitudinal direction. The end of the wrapping sheet 551F is folded once in such a manner as to wrap the interlabial pad 510, and the overlapped sides of the wrapping sheet 551F thus folded are bonded to each other by heat sealing or other method to form a pocket. The breaking line 553F for dividing the wrapping sheet 551F into two is provided outside from a closing tape 536F of the wrapping sheet 551F which closes the interlabial pad 510. The wrapping sheet 551F is further folded once or more times with its end covering the interlabial pad 510 disposed inside, and then the overlapped portions of the wrapping sheet 551F are closed. Examples of means for closing include the above-described closing tape 536F.

Additionally, the individual wrapping container 520F in this embodiment is different from the example of the fifth embodiment in that the individual wrapping container 520F is formed by a single sheet of the wrapping sheet 551F a part of which can be cut off. The individual wrapping container 520F illustrated in FIG. 29A can be cut along the breaking line 553F illustrated in FIG. 29B to obtain a separated part of the wrapping sheet 551F as illustrated in FIG. 29C. It is thus possible to divide the wrapping sheet 551F into a protective sheet 534F for covering the interlabial pad 510 and a discard sheet 550F used for discarding a used interlabial pad 511. Since the discard sheet 550F used for discarding the used interlabial pad 511 can be separated, the interlabial pad 510 before use can be held while packaged. Thus, the discard sheet 550F and the interlabial pad 510 held by one hand and the other, respectively, can be separated along the breaking line 553F, thereby preventing accidental dropping of the interlabial pad 510 before use.

In a modification of this embodiment, a folded end portion of a wrapping sheet 551H may be extended beyond a breaking line 553H toward the other end so as to form an accommodating space by the overlapped portions of the wrapping sheet 551H as illustrated in FIG. 30. The three sides of the overlapped portions of the wrapping sheet 551H may be sealed. This structure allows an accommodating space H to be provided also in an area of the wrapping sheet 551H where the interlabial pad 510 is not packaged.

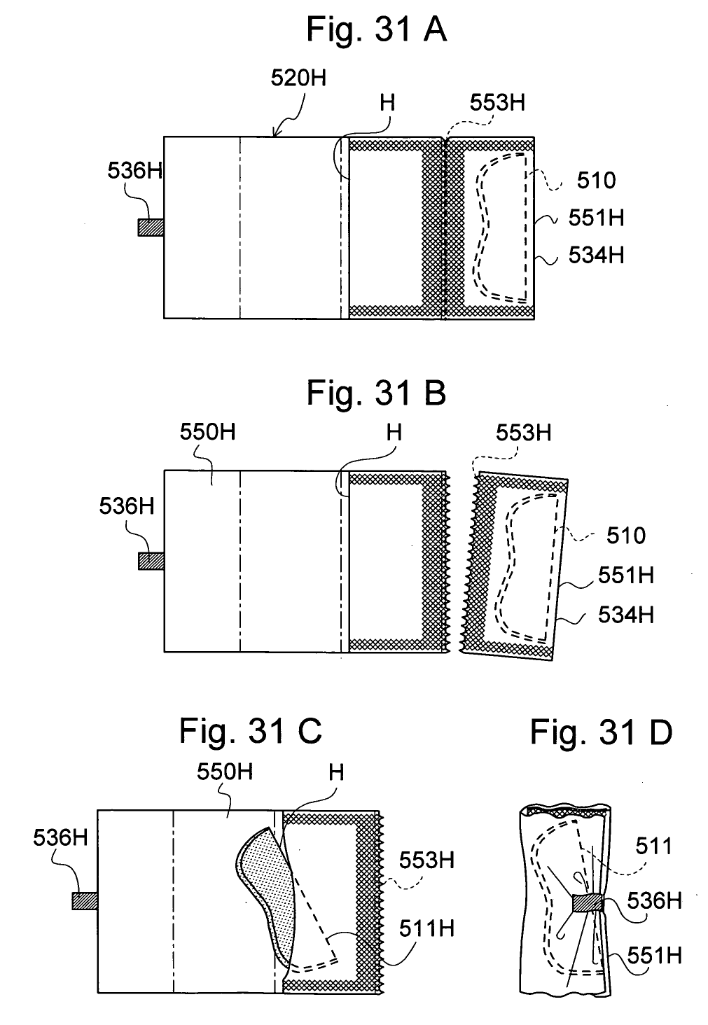

As illustrated in FIG. 31, an individual wrapping container 520H illustrated in FIG. 31A is cut along the breaking line 553H provided on the wrapping sheet 511H illustrated in FIG. 31B to obtain a protective sheet 534H and a discard sheet 550H. Subsequently, the used interlabial pad 511 is accommodated in the accommodating space H of the discard sheet 550H as illustrated in FIG. 31C. Then, the used interlabial pad 511 is enclosed in the discard sheet 550H by sealing an operable portion of the accommodating space H containing the used interlabial pad 511. Additionally, a closing tape 536H is provided at an end on a lateral side of the discard sheet 550H where the used interlabial pad 511 is not accommodated. The discard sheet 550H is rolled up with the side containing the used interlabial pad 511 inside, and is then closed by the closing tape 536H.

### Twelfth Embodiment

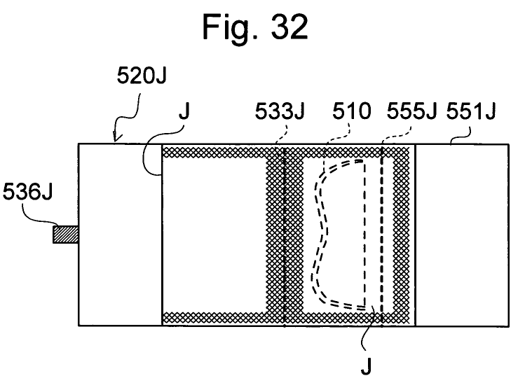

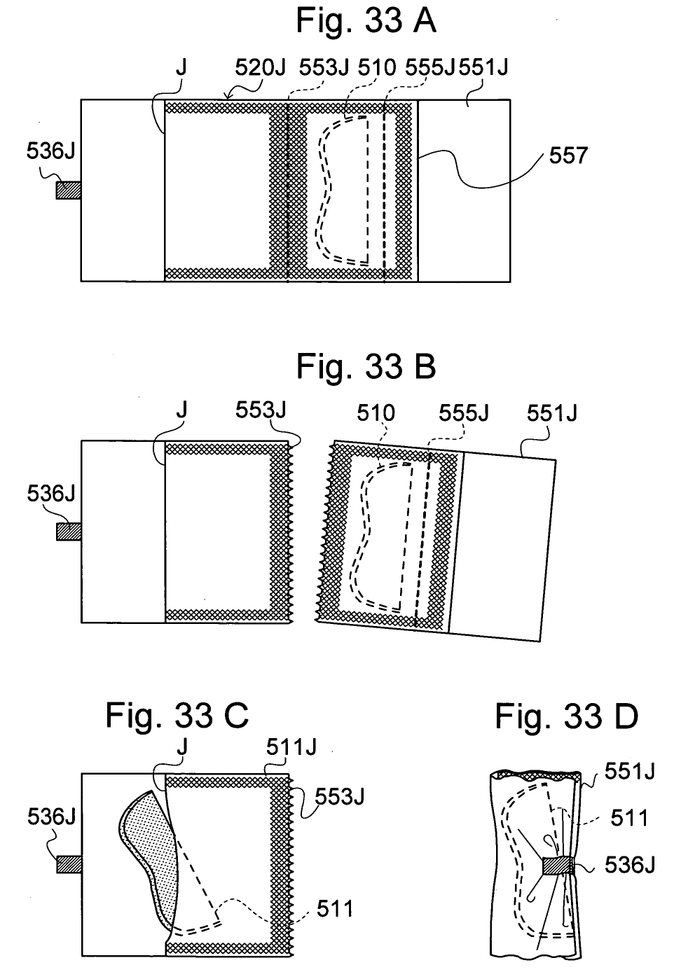

FIG. 32 is a front view of an unfolded individual wrapping container 520J in which the interlabial pad 510 is packaged in a twelfth embodiment of the present invention. FIG. 33A is a front view of the unfolded individual wrapping container 520J in which the interlabial pad 510 is packaged in the twelfth embodiment of the invention. FIG. 33B illustrates the individual wrapping container 520J in which the interlabial pad 510 is packaged in the twelfth embodiment of the invention with the individual wrapping container 520J broken along a breaking line 553J. FIG. 33C illustrates a condition where the used interlabial pad 511 is accommodated in a wrapping sheet 551J in the twelfth embodiment of the invention. FIG. 33D illustrates a method for discarding the used interlabial pad 511 in the twelfth embodiment of the invention.

In this embodiment, an additional mini-sheet 557 is overlapped on a single sheet of the wrapping sheet 551J to form accommodating spaces J by the mini-sheet 557. The individual wrapping container 520J illustrated in FIG. 33A is cut along a breaking line 553J provided on the wrapping sheet 551J. The interlabial pad 510 is inserted into either of the accommodating spaces J provided on the wrapping sheet 551J as illustrated in FIG. 33B. Then, the interiabial pad 510 is enclosed within the wrapping sheet 551J by sealing an unwrapped opening of the accommodating spaces J. Additionally, a closing tape 536J is provided at an end on a lateral side of the wrapping sheet 551J where the interlabial pad 510 before use is not accommodated as illustrated in FIG. 33C. The wrapping sheet 551J is rolled up with the side containing the used interlabial pad 511 inside, and is then closed by the closing tape 536J.

It is also possible to seal the mini-sheet 557 on the upper side so as to divide the encasement J into two.

Most Preferred Example of Interlabial Pad

It is preferable to fold the interlabial pad 510 in two along the longitudinal axis direction of the pad 510 with the surface side of the pad material disposed on the convex side, and wrap the two-folded pad 510 in the protective sheet 534. Since the two-folded interlabial pad 510 is disposed such that its convex side faces to the folded portion of the protective sheet 534 on the lower side of the overlapped regions, the back side of the pad material appears on the unwrapped opening side when the protective sheet 534 is opened. The reason for folding the interlabial pad 510 double is that the interlabial pad 510 is attached in a two-folded condition where its surface side faces to the inner surface of the labia while two parts of the back side face to each other. The interlabial pad 510 which is folded in advance with the surface side of the pad material disposed on the convex side can be taken out of the protective sheet 534 and attached without touching the surface side of the interlabial pad 510 by hand. Conversely, if the interlabial pad 510 is packaged in a flat condition without fold or in a folded with its back side disposed on the outside, an action for disposing the surface side of the pad material on the outside is required after taking out the interlabial pad 510. In this case, there is a possibility of contamination of the interlabial pad 510 before use by touching the surface side of the interlabial pad 510 by fingers when the interlabial pad 510 is folded to dispose the surface side of the pad material on the outside. However, such contamination is prevented in advance according to the present invention.

Dimensions of Interlabial Pad and Protective Sheet

The apparent length of the interlabial pad is preferably in a range from 50 to 160 mm in the longitudinal direction and in a range from 10 to 60 mm in the lateral direction. The length of the protective sheet covering the interlabial pad is only required to be equal to or larger than the length of the interlabial pad 510. More specifically, the length of the protective sheet is in a range from 75 to 200 mm in the vertical axis direction and in a range from 30 to 300 mm in the horizontal direction.

Structures of Protective Sheet and Discard Sheet

The outer surface of the protective sheet in which the interlabial pad is sealed is bonded to the discard sheet to form a structure. The bonded portions of the protective sheet and the discard sheet can be released and separated from each other. More specifically, adhesive material is provided substantially at the center of the outer surface of the protective sheet where the unwrapped opening is not formed so as to bond the protective sheet to the discard sheet. Hot melt in an amount of 5 to 10 g/m² is applied to the adhesive area, which is the same as the above described adhesive material example. The portion of the discard sheet for receiving the adhesive material is made from a releasable material, or separating treatment is given to the discard sheet. The discard sheet is so sized as to be sufficient for wrapping the used interlabial pad for discard, and is folded and closed by the closing tape. The discard sheet may be folded in pleats or rolled up. The closing tape is the same as in the above-described example. The method for bonding the protective sheet and the discard sheet is not limited to the use of the adhesive material, but may be heat sealing and mating embossment. However, it is more preferable to employ adhesive material by which the protective sheet can be attached to any place in the lavatory for discarding the used interlabial pad using the discard sheet. In this structure, the interlabial pad is sealed in the protective sheet. If not sealed, the interlabial pad is dropped out while being carried by the wearer. The structure of the invention makes it possible to temporarily fix the interlabial pad sealed in the protective sheet to somewhere and then unfold the discard sheet after the interlabial pad and the discard sheet are separated.

When the discard sheet is separated from the protective sheet, the discard sheet folded in a compact size is not obstructive, thereby enhancing handling easiness. However, since the portion from which the protective sheet and the discard sheet are released and separated (the portion where those sheets are not bonded to each other by the adhesive material) is exposed, the protective sheet and the discard sheet may be released and separated while being carried by the wearer. There are other examples in which: the discard sheet is bonded to the inner surface of the protective sheet and sealed together with the interlabial pad; and the discard sheet and the interlabial pad are both sealed within the protective sheet in addition to the above-described example. However, it is not preferable to adopt these structures since the interlabial pad before use may be dropped from the protective sheet when the protective sheet is opened to take out the discard sheet.

Material for Protective Sheet

The protective sheet is made from a sheet having high barrier property. Examples of such material involve polyethylene, polypropylene, polyester, polyvinyl alcohol (PVA), polylactate, polybutylene succinate, nonwoven fabrics and paper, and laminated material thereof, each having a thickness of 15 to 60 μm. Antimicrobial treatment may be applied to these materials. Alternatively, the protective sheet may be formed by laminated tissue made of a pulp having a basis weight per unit area of 10 to 25 g/m² and a thickness of 0.05 to 0.2 mm coated with 10 to 30 μm of PVA. More specifically, a polyethylene film having a basis weight per unit area of 25 g/m² may be employed. It is more preferable that the protective sheet is made from material which can be thrown into a flush lavatory. For this purpose, the protective sheet is constituted by biodegradable material, water soluble material, and water dispersive material. This structure prevents contaminants such as dirt, dust and germs from contacting with the surface of the interlabial pad which contacts the inner surface of the labia at the time of temporarily placing the interlabial pad before use covered by the protective sheet so as to when the used interlabial pad is detached and discarded using the discard sheet, thereby avoiding secondary infection.

The “biodegradable” property refers to degradation of a substance to gas such as carbon dioxide or methane, water and biomass under a condition anaerobic or aerobic condition in the existence of germs such as actinomyces and other micro-organisms according to the processes in nature. The biodegrading property (biodegradation speed, biodegradation degree etc.) of the substance concerned is equal to that of material produced naturally such as a fallen leave or of a synthetic polymer commonly recognized as biodegradation under the same environmental condition. The “water dispersive” property is similar to hydrolysable property, in which fibers are easily dispersed into fragments at least small enough not to block piping of an ordinary lavatory in a large amount of water or water flow, though the dispersion does not occur in a limited amount of water (such as menstrual blood) during use. The “water soluble” property refers to a soluble property in a large amount of water or water flow, though the solubility does not occur in a limited amount of water (such as menstrual blood) during use.

Material for Discard Sheet

The discard sheet is made from material having high liquid-barrier property. This property prevents leakage of menstrual blood adhering to or absorbed by the used interlabial pad from the discard sheet, and allows the used interlabial pad to be wrapped in the discard sheet and discarded without contaminating the wearer's hands.

Adhesive Material

The adhesive material is formed by applying hot melt in a basis weight per unit area from 5 to 10 g/m². The application pattern of the adhesive material may be selected from dots, lines, waves, spirals etc. Dots which are 1 to 3 mm in diameter are preferably applied to 30 to 70% of one side of the protective sheet. The adhesive material thus provided allows the inside wrapping body to be temporarily fixed on a wall or a paper holder without dropping of the interlabial pad due to tilting or shock, and to be easily removed from the temporary fixing position.

With respect to the region to which the adhesive material is applied, the protective sheet has a first region and a second region on a side where the protective sheet does not contact the interlabial pad, and the adhesive material is applied to either or both of the first and second regions. A region of at least 20 by 20 mm square where the adhesive material is not applied is provided on both surfaces of the protective sheet. This region secures a portion for holding the protective sheet covering the interlabial pad (a portion touched by the thumb and the forefinger) by the wearer, and prevents adhesion of the adhesive material on the protective sheet to the hand and resultant difficulty in handling of the protective sheet when the protective sheet is held and temporarily placed or the interlabial pad is taken out of the protective sheet.

The adhesive material is required to have adhesion sufficient for adhering to a wall, a paper holder or other place. For example, the adhesive material is obtained by fusion-mixing synthetic rubber resin as a chief component such as styrene-isoprene-styrene block copolymer (SIS), styrene-butadiene-styrene block copolymer (SBS), styrene-ethylene/butadiene-styrene block copolymer (SEBS), styrene-ethylene/propylene-styrene block copolymer (SEPS) with tackifier such as terpene resin and rosin resin and plasticizer such as wax. More specifically, adhesive material formed by fusion-mixing 15 to 25% of SEBS, 15 to 35% of plasticizer and 40 to 70% of tackifier may be employed. In the pressure sensitive type hot melting, 0.1 to 1.0% of antioxidant, anti-fluorescent agent or other may be added.

Closing Tape

The closing tape may be formed by a film to which adhesive is applied on one side, by tissue to which adhesive is applied on one side so as to be thrown into a flush lavatory or by other known material, and is not limited to those.

Temporary Closing Member

The temporary closing member is made to be releasable to such a degree that the inner wrapping body is not damaged when the temporary closing member is released by applying release treatment to the protective sheet or affixing a release-treated material. The material to be release-treated may be selected from thin paper, nonwoven fabrics, laminated paper and a film. The releasing agent is preferably made from the silicone resin family, the fluororesin family or the octadecyl isocyanate family. More specifically, a sheet formed by thin paper coated with silicone resin may be used. For affixing the release-treated material to the protective sheet, the release-treated material is disposed such that the release-treated side faces to the protective sheet and the non-treated side faces to the discard sheet, and is affixed by the same material as used for the adhesive material.

Structure, Strength and Starting Position of Breaking Line

The breaking line is formed by alternately disposing breakable portions and unbreakable portions in the form of sawing machine stitches, for example. It is preferable to determine the length of the breakable portion in a range from 0.5 to 5 mm, the width thereof in a range of 3 mm or smaller, and the length of the unbreakable portion in a range from 0.5 to 3 mm. The breaking strength of the breaking line is preferably 0.2 to 3.0N/25 mm, more preferably 0.3 to 1.5N/25 mm when pulled in a direction perpendicular to the breaking line which is disposed at the center under a pulling condition of 25 mm width and 100 m/min. For visually indicating the starting position of the breaking line for the wearer, an arrow may be printed or a cutting shape (rounded end etc.) different from the shape at the end position may be provided in the vicinity of the starting position of the breaking line.

C. Outside Wrapping Container and Intermediate Wrapping Container for Protecting Individual Wrapping Body

### Thirteenth Embodiment

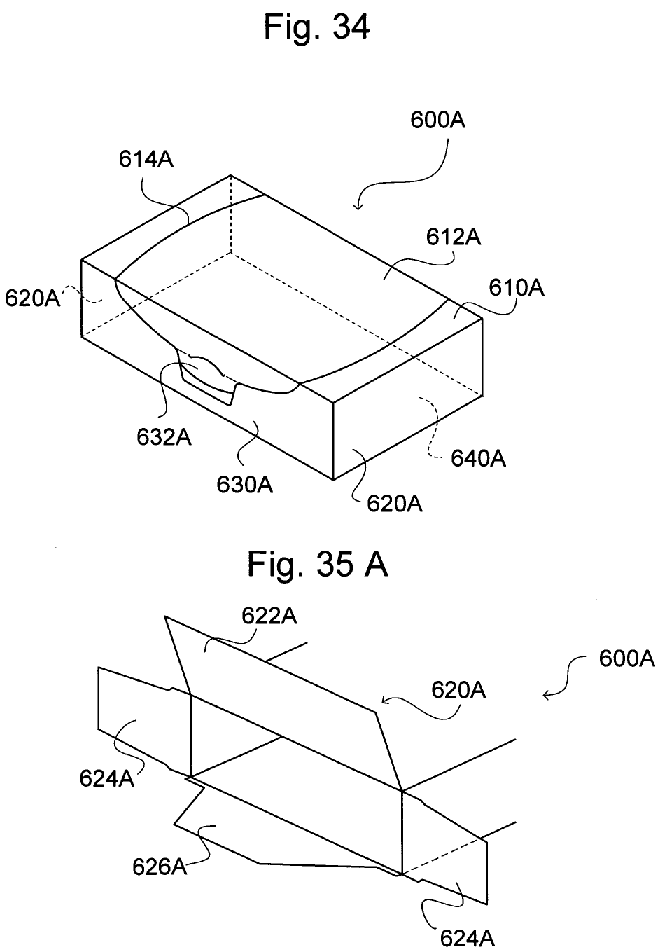

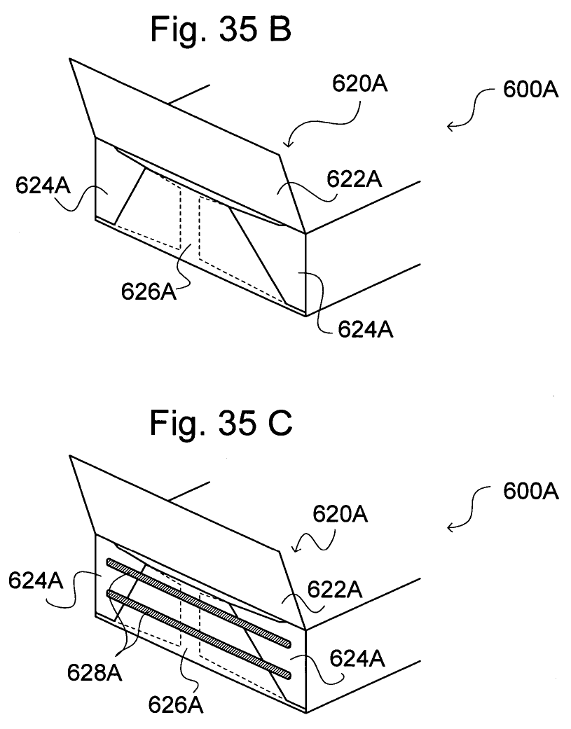

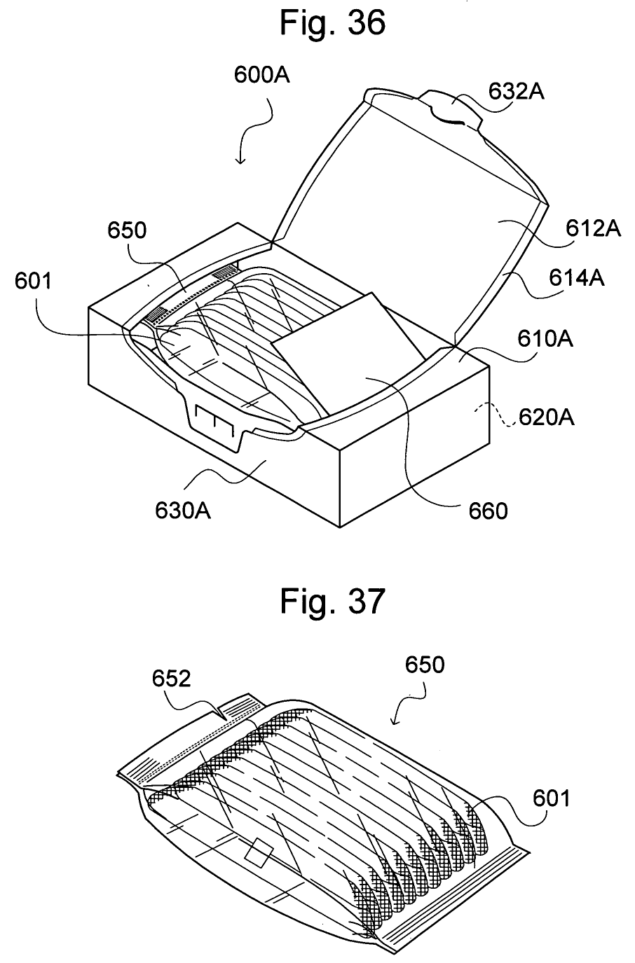

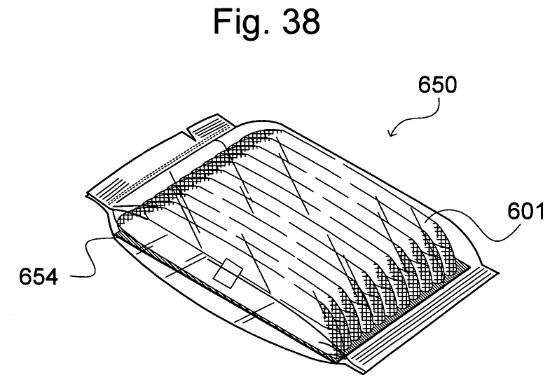

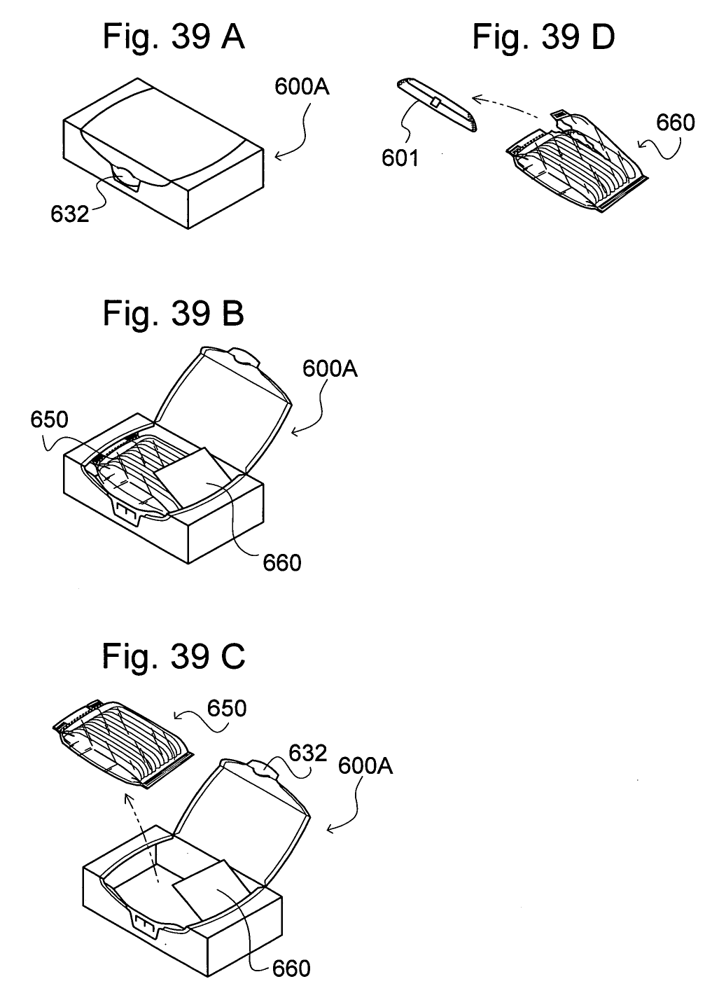

FIG. 34 is a perspective view of an outer wrapping container 600A in a thirteenth embodiment of the present invention. FIGS. 35A through 35C illustrate steps for assembling a side face 620A of the outer wrapping container 600A in the thirteenth embodiment of the invention. FIG. 36 illustrates a condition where a lid 612A of the outer wrapping container 600A is opened in the thirteenth embodiment of the invention. FIG. 37 is a perspective view of an intermediate wrapping container 650 in the thirteenth embodiment of the invention. FIG. 38 is a perspective view showing an example in which a reinforcing sheet is inserted into the inside of the intermediate wrapping container 650. FIGS. 39A through 39D illustrate steps until taking an individual wrapping body 601 out of the outer wrapping container 600A in the thirteenth embodiment of the invention.

As illustrated in FIG. 34, the outer wrapping container 600A includes an upper face 610A, a lower face 640A, the side faces 620A, and a front face 630A. The outer wrapping container 600A also includes a cutting 614A which extends from an edge of the upper face 610A through the front face 630A to another edge of the upper face 610A. The cutting 614A provides the openable and closable lid 612A on the front face 630A and the upper face 610A as illustrated in FIG. 34. Additionally, an opening portion 632A is provided at the tip of the lid 612A on the front face 630A side.

The cutting 614A is so formed as to extend from one edge of the upper face 610A through the front face 630A to another edge of the upper face 610A by a so-called half-cutting method which does not produce serrated edges like sawing machine stitches when opened. The half-cutting method also allows the separable portion of the lid 612A to be connected with the outer wrapping container 600A without adhesive.

As illustrated in FIGS. 35A through 35D, each of the side faces 620A of the outer wrapping container 600A includes an upper end 622A, side ends 624A, and a lower end 626A.

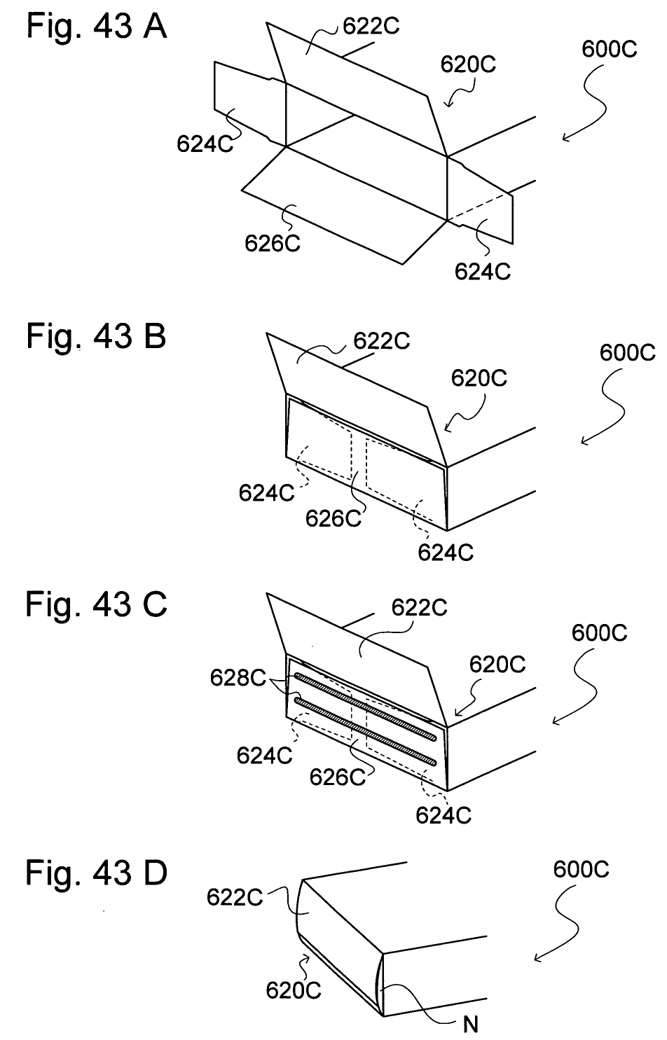

Shown in FIG. 43A are steps for assembling a side face 620C of a conventional outer wrapping container 600C. First, side ends 624C are folded inside as illustrated in FIG. 43A. Next, a lower end 626C is folded inside in such a manner as to overlie the side ends 624C as illustrated in FIG. 43B. Then, adhesive 628C is applied onto the upper surface of the lower end 626C. Finally, the upper end 622C is folded inside in such a manner as to overlie the adhesive 628C applied onto the lower end 626C as illustrated in FIG. 43C. In the side face 620C of the outer wrapping container 600C thus assembled, no adhesive is applied between the side ends 624C and the lower end 626C as illustrated in FIG. 43D. Thus, a clearance “x” tends to exist, which may lower the strength of the outer rapping container 600C and permit dust and dirt to enter into the outside wrapping container 600C.

Now, steps for assembling the side face 620A of the outer wrapping container 600A in this embodiment are described with reference to FIGS. 35A through 35C. First, the side ends 624A of the outer wrapping container 600A illustrated in FIG. 35A are folded inside as illustrated in FIG. 35B, and subsequently the lower end 626A is folded inside in such a manner as to overlie the folded side ends 624A. The lower end 626A in this embodiment is cut in the trapezoid shape such that adhesive 628A can be applied to both of the two side ends 624A which are exposed. Then, the adhesive 628A is applied to the lower end 626A as illustrated in FIG. 35C. Finally, the upper end 622A is folded inside in such a manner as to overlie the adhesive 628A applied onto the lower end 626A. In the side face 620A of the outer wrapping container 600A thus assembled, the side ends 624A and the upper end 622A are bonded by adhesive, thereby preventing a clearance from being produced. Thus, entrance of dust and dirt into the outer wrapping container 600C is prevented while increasing the strength of the outer wrapping container 600A against pressures from above and below.

In this embodiment, the intermediate wrapping container 650 and an instruction manual 660 appear when the lid 612A is opened using the opening portion 632A of the outer wrapping container 600A as illustrated in FIG. 36. It is preferable to dispose the instruction manual 660 in such a position that the wearer can recognize it without fail as in this embodiment. This arrangement ensures that the wearer reads the manual. The intermediate wrapping container 650 is accommodated in the outer wrapping container 600A such that space is produced between the intermediate wrapping container 650 and the outer wrapping container 600A. This space prevents crush of the intermediate wrapping container 650 when external pressure is applied to the outer wrapping container 600A, since the external pressure is absorbed by the deformation of the outer wrapping container 600A.

As illustrated in FIG. 37, the individual wrapping bodies 601 are packaged in the intermediate wrapping container 650. The intermediate wrapping container 650 is preferably filled with air since it is completely sealed. This structure prevents transmission of shock to the interlabial pad, especially the peripheral portion of the pad accommodated in the individual wrapping body 601 when the wearer accidentally drops the intermediate wrapping container 650, since the air within the intermediate wrapping container 650 absorbs the shock. Thus, the interlabial pad contained in the individual wrapping body 601 can be attached without degradation of wearing comfortableness even if the wearer accidentally drops the intermediate wrapping container 650. The intermediate wrapping container 650 also includes a cutting 652 for opening the intermediate wrapping container 650. The cutting 652 allows the intermediate wrapping container 650 to be easily opened by hands.

The number of the individual wrapping bodies 601 sealed within the intermediate wrapping container 650 is preferably 12. However, it is only required to be at least two, and is not specifically limited.

Additionally, a feature of the intermediate wrapping container 650 is that it is made from a non-breathable material. This prevents entrance of moisture from the outside to the inside of the intermediate wrapping container 650, thereby obtaining an almost stable amount of moisture in the intermediate wrapping container 650 regardless of outside environmental changes. Furthermore, since the individual wrapping body 601 is covered by the intermediate wrapping container 650 made from a non-breathable material, it is prevented that the original absorption ability of the interlabial pad which is chiefly composed of a water absorption material such as the small cellulose family and accommodated in the individual wrapping body 601 is degraded, due to moisture absorption by the interlabial pad before attachment, and that the rigidity of the interlabial pad itself is extremely lowered, especially the rigidity of the peripheral portion thereof. Accordingly, since moisture inside the intermediate wrapping container 650 is almost kept constant, the rigidity of the interlabial pad accommodated in the individual wrapping body 601, especially rigidity of the peripheral portion thereof is not adversely affected with ease by weather variations or accidental dropping of the intermediate wrapping container 650 containing a plurality of the individual wrapping bodies 601 in the lavatory by the wearer. Thus, the interlabial pad packaged in the individual wrapping body 601 having stable quality can be provided for the wearer.

As illustrated in FIG. 38, the intermediate wrapping container 650 may include a reinforcing sheet 654 so as to protect at least a part of the individual wrapping bodies 601 sealed in the intermediate wrapping container 650. The reinforcing sheet 654 is preferably provided inside or outside the intermediate wrapping container 650 to protect at least a part of a plurality of the individual wrapping bodies 601. More specifically, the reinforcing sheet 654 may be disposed in such a manner as to cover the lower surfaces of the plural individual wrapping bodies 601, to cross over at least one face of each individual wrapping body 601, or to cover the entire surfaces of the individual wrapping bodies 601 with an openable region provided therein, for example, when the reinforcing sheet 654 is included inside the intermediate wrapping container 650. On the other hand, the reinforcing sheet 654 is connected to the intermediate wrapping container 650 by adhesive, for example, when the reinforcing sheet 654 is provided outside the intermediate wrapping container 650.

A plurality of the reinforcing sheets 654 may be provided, or a single sheet of the folded reinforcing sheet 654 may be included. Examples of material for the reinforcing sheet 654 involve paper, a heavy paper, a plastic, polyethylene, polypropylene, polyethylene terephthalate, polylactate, polybutyl succinate, nonwoven fabrics and paper, and laminated material thereof. Additionally, instructions on how to use the interlabial pad may be printed on the reinforcing sheet 654 with ink or other means. More specifically, a company name or brand name may be printed with ink or other means. It is also allowed to mix pigment in a range from 0.2 to 10% or to print the reinforcing sheet 654 with ink or others so as to make it difficult to visually recognize the interlabial pad itself contained in the intermediate wrapping container 650. Furthermore, instructions on how to open the individual wrapping body 601 or how to use the interlabial pad contained in the intermediate wrapping container 650 may be printed on the reinforcing sheet 654 with ink or other means.

FIGS. 39A through 39D illustrate steps until taking the interlabial pad contained in the individual wrapping body 601 out of the outer wrapping container 600A in this embodiment. When the lid 612A is opened using the opening portion 632A of the outer wrapping container 600A as illustrated in FIG. 39A, the inside of the outer wrapping container 600A containing the intermediate wrapping container 650 and the instruction manual 660 appears, which can be visually recognized as illustrated in FIG. 39B. Then, the intermediate wrapping container 650 is taken out of the outer wrapping container 600A as illustrated in FIG. 39C. Finally, the individual wrapping body 601 sealed in the intermediate wrapping container 650 is taken out.

### Fourteenth Embodiment

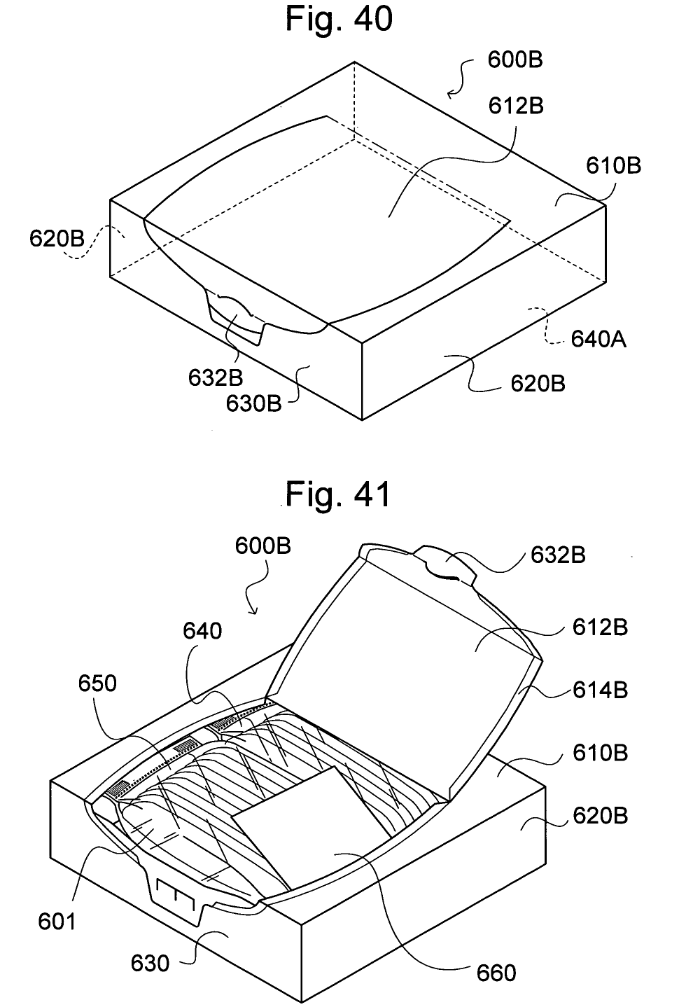

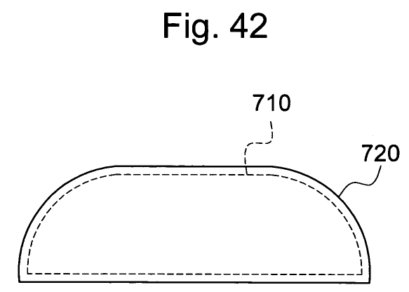

FIG. 40 is a perspective view of an outer wrapping container 600B in a fourteenth embodiment of the present invention. FIG. 41 illustrates a condition where a lid 612B of the outer wrapping container 600B is opened in the fourteenth embodiment of the invention.

In this embodiment, the outer wrapping container 600B is different from the outer wrapping container 600A in the thirteenth embodiment in that a cutting 614B extends from a middle portion of an upper face 610B through a front face 630B to another middle portion of the upper face 610B with a fold provided as illustrated in FIG. 40, and that a pair of the intermediate wrapping containers 650 are accommodated in the outer wrapping container 600B as illustrated in FIG. 41.

The number of the intermediate wrapping containers 650 accommodated in the outer wrapping container 600B is preferably one or two. However, it is only required to be at least one, and is not specifically limited.

Dimensions of Outer Wrapping Container

The volume of the outer wrapping container is required to be larger than at least the entire volume of the plural individual wrapping bodies or the volume of the intermediate wrapping container covering those. More specifically, the length of the outer wrapping container is 0 to 80 mm, preferably 0 to 40 mm larger than the entire length of the plural individual wrapping bodies or the length of the intermediate wrapping container covering those. The width of the outer wrapping body is 0 to 80 mm, preferably 0 to 40 mm larger than the entire width of the plural individual wrapping bodies or the width of the intermediate wrapping container covering those. The height of the outer wrapping body is 0 to 80 mm, preferably 0 to 40 mm larger than the entire height of the plural individual wrapping bodies or the height of the intermediate wrapping container covering those.

Material for Outside Wrapping Container

Examples of material for the outer wrapping container include paper, plastic, laminated paper, and aluminum-evaporated paper.

Dimensions of Intermediate Wrapping Container

The volume of the intermediate wrapping container is required to be larger than at least the entire volume of the plural individual wrapping bodies. More specifically, the length of the intermediate wrapping container is 0 to 80 mm, preferably 0 to 40 mm larger than the entire length of the plural individual wrapping bodies. The width of the intermediate wrapping body is 0 to 80 mm, preferably 0 to 40 mm larger than the entire width of the plural individual wrapping bodies. The height of the intermediate wrapping body is 0 to 80 mm, preferably 0 to 40 mm larger than the entire height of the plural individual wrapping bodies. The proportion of air contained within the intermediate wrapping container is in a range from 5 to 60%, preferably in a range from 20 to 40%. More specifically described, the length, width and height of the intermediate wrapping container are 185 mm, 120 mm and 50 mm, respectively, when the entire length, width and height of the individual wrapping bodies are 135 mm, 85 mm and 35 mm, respectively. The proportion of contained air in this case is 36%.

Material for Intermediate Wrapping Container

Material used for the intermediate wrapping container is not specifically limited as long as it is non-moisture-permeable material. Examples of such material involve polyethylene, polypropylene, polyethylene terephthalate, polylactate, polybutyl succinate, laminated nonwoven fabrics and paper, and aluminum-evaporated nonwoven fabrics and paper, each of which is 15 to 80 μm in thickness. More specifically, a film chiefly made from high density polyethylene (HDPE) resin in an adjusted weight of 30 to 60 g/m² may be employed. Additionally, a drawn or embossed film for enhancing resin orientation and thereby increasing its rigidity may be used.

The present invention can be utilized as an individual wrapping container capable of preventing deformation and damage of a peripheral portion of a interlabial pad.

1. An individual wrapping container in which an interlabial pad attached between labia is packaged, wherein
the interlabial includes a peripheral portion forming a contour of the interlabial pad; and the individual wrapping container includes a protective region having higher rigidity than the rigidity of the peripheral potion of the interlabial pad so as to protect the peripheral portion.

2. The individual wrapping container according to claim 1, wherein
the peripheral portion of the interlabial pad includes a vestibular floor contact region contacting with a vestibular floor of a wearer during attachment of the interlabial pad; and
the protective region is disposed at least in a portion where the individual wrapping container contacts the vestibular floor contact region when the interlabial pad is packaged in said individual wrapping container.

3. The individual wrapping container according to claim 1, wherein the protective region is made from a rigid material having predetermined rigidity.

4. The individual wrapping container according to claim 1, wherein the protective region is embossed.

5. The Individual wrapping container according to claim 1, wherein: the individual wrapping container is constituted by a sheet body; and the protective region is formed in a folded portion formed by folding a part of the sheet body.

6. The individual wrapping container according to claim 1, wherein a buckling strength of the protective region is established In a range from 100 mN to 2,000 mN.

7. An individual wrapping body comprising the individual wrapping container according to claim 1 and an interlabial pad accommodated in the individual wrapping container, wherein
the interlabial pad includes a peripheral portion forming a contour of the interlabial pad; and
a buckling strength of the peripheral portion of the interlabial pad is established in a range from 50 mN to 400 mN.

8. The individual wrapping container in which an interlabial pad attached between labia is packaged, comprising a protective sheet for preventing contamination of the interlabial pad before use, and a discard sheet used for discarding a used interlabial pad, wherein
the protective sheet directly covers the interlabial pad before use to form an inner wrapping body, and the discard sheet covers the inner wrapping body when the interlabial pad is packaged in the individual wrapping container; and
a temporary closing member for releasably fixing the inner wrapping body and the discard sheet is provided on a surface where the inner wrapping body contacts the discard sheet and/or a surface where the discard sheet contacts the inner wrapping body.

9. The individual wrapping container according to claim 8, wherein the temporary closing member is releasable to such a degree that the inner wrapping body is not damaged when the inner wrapping body is separated from the discard sheet, and also has sufficient adhesion for re-adhering after separation.

10. An individual wrapping container in which an interlabial pad attached between labla is packaged, wherein
the individual wrapping container is constituted by a wrapping sheet having a breaking line along which a part of the wrapping sheet can be separated; and
the part separated along the breaking line becomes a discard sheet used for discarding a used interlabial pad.

11. The individual wrapping container according to claim 10, wherein: the individual wrapping container includes at least an accommodating space for accommodating an interlabial pad before use and/or a used interlabial pad; and
the accommodating space is formed by folding a part of the wrapping sheet where the breaking line is not located.

12. The individual wrapping container according to claim 10, wherein
the individual wrapping container includes at least an accommodating space for accommodating an interlabial pad before use and/or a used interlabial pad; and
the accommodating space is a space formed between the wrapping sheet and a mini-sheet attached to the wrapping sheet.

13. A wrapping body comprising:
individual wrapping bodies each including the individual wrapping container according to claim 1 and an interlabial pad packaged in the individual wrapping container, and an outer wrapping container for accommodating a plurality of the individual wrapping bodies, wherein
a subdividing intermediate wrapping container for accommodating the two or more individual wrapping bodies to be sealed therein is provided within the outer wrapping container.

14. A wrapping body comprising:
individual wrapping bodies each including the individual wrapping container according to claim 8 and an interlabial pad packaged in the individual wrapping container, and an outer wrapping container for accommodating a plurality of the individual wrapping bodies, wherein
a subdividing intermediate wrapping container for accommodating the two or more individual wrapping bodies to be sealed therein is provided within the outer wrapping container.

15. A wrapping body comprising:
individual wrapping bodies according to claim 7 and an interiablal pad packaged in the individual wrapping container, and an outer wrapping container for accommodating a plurality of the individual wrapping bodies, wherein
a subdividing intermediate wrapping container for accommodating the two or more individual wrapping bodies to be sealed therein is provided within the outer wrapping container.

16. The wrapping body according to claim 13, wherein the intermediate wrapping container is disposed such that a clearance is provided between the intermediate wrapping container and an inner wall of the outer wrapping container.

17. The wrapping body according to claim 13, wherein the intermediate wrapping container is made from an airtight material so that the individual wrapping bodies can be airtight-sealed therein.
